SEQUENCE LISTING (15 sequences)

SEQ ID NO: 1 - DNA, 54 bp, Artificial Sequence
  Synthetic Construct
cggtcgagctattgaaagctagctagttgtccttgtctacctgggtgcgcacga

SEQ ID NO: 2 - DNA, 20 bp, Artificial Sequence
  Synthetic Construct
agctagctttcaatagctcg

SEQ ID NO: 3 - DNA, 20 bp, Artificial Sequence
  Synthetic Construct
cctacactgggataattgac

SEQ ID NO: 4 - DNA, 24 bp, Artificial Sequence
  Synthetic Construct
accggtcaattatcccagtgtagg

SEQ ID NO: 5 - DNA, 36 bp, Artificial Sequence
  Synthetic Construct
tttttttcgtgcgcacccaggtagacaaggacaact

SEQ ID NO: 6 - DNA, 36 bp, Artificial Sequence; identical to SEQ ID NO: 5
  Synthetic Construct

SEQ ID NO: 7 - DNA, 51 bp, Artificial Sequence
  Synthetic Construct
tatcaacagtgaaccatattttcgtgcgcacccaggtagacaaggacaact

SEQ ID NO: 8 - DNA, 51 bp, Artificial Sequence
  Synthetic Construct
agccagcagagacacacgttttcgtgcgcacccaggtagacaaggacaact

SEQ ID NO: 9 - DNA, 51 bp, Artificial Sequence
  Synthetic Construct
ggcccgcagcgaccacccttttcgtgcgcacccaggtagacaaggacaact

SEQ ID NO: 10 - DNA, 49 bp, Artificial Sequence
  Synthetic Construct
ttttttctctggttaacgtgtctgggcattttatggttcactgttgata

SEQ ID NO: 11 - DNA, 49 bp, Artificial Sequence
  Synthetic Construct
ttttttctctggttaacgtgtctgggcatttcgtgtgtctctgctggct

SEQ ID NO: 12 - DNA, 49 bp, Artificial Sequence
  Synthetic Construct
ttttttctctggttaacgtgtctgggcatttgggtggtcgctgcgggcc

SEQ ID NO: 13 - DNA, 42 bp, Artificial Sequence
  Synthetic Construct
cgcgcgtacttttcgtgcgcacccaggtagacaaggacaact

SEQ ID NO: 14 - DNA, 40 bp, Artificial Sequence
  Synthetic Construct
ttttttctctggttaacgtgtctgggcatttgtacgcgcg

SEQ ID NO: 15 - DNA, 22 bp, Artificial Sequence
  Synthetic Construct
tgcccagacacgttaaccagag

(19) United States
(12) Patent Application Publication
(10) Pub. No.: US 2024/0416338 A1
(43) Pub. Date: Dec. 19, 2024

(54) MULTIPLEXED MICROFLUIDIC FORCE SPECTROSCOPY ON-A-CHIP

(71) Applicant: Ohio State Innovation Foundation, Columbus, OH (US)

(72) Inventors: Ehsan AKBARI, Columbus, OH (US); Melika SHAHHOSSEINI, Columbus, OH (US); Carlos CASTRO, Powell, OH (US); Jonathan SONG, Bexley, OH (US)

(21) Appl. No.: 18/274,082

(22) PCT Filed: Jan. 26, 2022
(86) PCT No.: PCT/US2022/013819

§ 371 Date: Jul. 25, 2023

Related U.S. Application Data
(60) Provisional application No. 63/141,625, filed on Jan. 26, 2021.

(51) Int. Cl. B01L 3/00; G01N 15/14

(52) U.S. Cl. CPC B01L 3/502715; B01L 3/502707; G01N 15/1484; B01L 2200/027; B01L 2200/12; B01L 2300/12

(57) ABSTRACT

Disclosed herein is a device, system, method of using the device, and method of manufacturing the device, wherein the device comprises multiplexed, microfluidic force spectroscopy on a chip.

### CROSS-REFERENCE TO RELATED APPLICATIONS

This application claims benefit of U.S. Provisional Application No. 63/141,625, filed Jan. 26, 2021, incorporated herein by reference in its entirety.

### GOVERNMENT SUPPORT CLAUSE

This invention was made with government support under R01 HL141941 awarded by the National Institutes of Health. The government has certain rights in the invention.

### BACKGROUND

Force spectroscopy enables controlled mechanical manipulation of single molecules and probing of molecular interactions, which can reveal significant insights in biomolecular mechanobiology and biomedical engineering. Advances in molecular biophysics have been propelled by several force spectroscopy techniques such as magnetic tweezers, atomic force microscopy, and optical tweezers. (Ha et al.; Roy et al.) However, these techniques require the use of complex, specialized, and often cost-prohibitive instrumentation for most laboratories (McDonald et al.) that are often very low-throughput, usually testing one or a few interactions at a time. Most force spectroscopy techniques require a highly specialized set of equipment for manipulation and imaging, which leads to high instrumentation cost and bulky equipment, making single molecule force spectroscopy unavailable to most laboratories.

Recent developments in centrifugal force microscopy, (Kim et al.) acoustic bead manipulation, (Strick et al. 1996; Strick et al. 1998) wide-range magnetic field, (Guizar-Sicairos et al.) and nanophotonic bead trapping (Evans et al. 1997; Evans 2001) have helped reduce the total cost per force spectroscopy experiment by enabling multiplexed manipulation and loading of molecular constructs. Despite recent efforts such as centrifugal force microscopy to reduce the cost and increase the throughput of force spectroscopy, there is room for further cost reduction, simplification, portability, and accessibility. Therefore, it would be a significant benefit to the fields of molecular biophysics, mechanobiology, and bioengineering to develop a low-cost, high-throughput force spectroscopy setup that enables single molecule manipulation of molecular interactions.

### SUMMARY

The present disclosure provides a technique that addresses the challenges with single molecular force spectroscopy, as discussed above. More specifically, this disclosure uses microfluidic technology to enable multiplexed mechanical manipulation and testing of molecular complexes and binding interactions.

Thus, in one aspect the present disclosure provides for a microfluidic device for measuring hydrodynamic force between two components, wherein the device comprises: a rigid material, wherein said rigid material comprises at least two channels of varying width that are serially connected; and a first component, wherein said first component is functionally attached to a second component, and further wherein the second component is anchored to the rigid material.

In another aspect, the present disclosure provides for a system for measuring molecular interactions between molecules, the system comprising a microfluidic device comprising a rigid material, wherein said rigid material comprises at least two channels of varying width that are serially connected; and a first component, wherein said first component is functionally attached to a second component, and further wherein the second component is anchored to the rigid material, wherein fluid can be forced through the channels, and further wherein hydrodynamic force between the first component and the second component can be measured.

In a further aspect, the present disclosure provides for a method of determining strength of molecular interactions between a first molecule and a second molecule comprising: providing a device, wherein the device comprises (i) a rigid material, wherein said rigid material comprises at least two channels of varying width that are serially connected; (ii) a first component with the first molecule associated therewith; and (iii) a second component, with a second molecule associated therewith; wherein the first component and the second component can associate with each other through the first and second molecules; executing at least one force spectroscopy test; and measuring dissociation force between the first and the second molecule, thereby determining the strength of the molecular interaction between the first and the second molecule.

Lastly, in another aspect the present disclosure provides for a method of manufacturing a device, the method comprising designing a network of at least two channels; producing transparencies; using the transparencies as masks in photolithography to spin-coat the network of at least two channels onto a wafer to create a microfluidic platform with at least two channels; placing at least one post on the design of at least two channels to form at least one reservoir; casting a prepolymer onto the microfluidic platform; curing the prepolymer; removing the prepolymer from the microfluidic platform; oxidizing the prepolymer in a plasma discharge; and attaching the prepolymer to a rigid material.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. The application includes reference to the accompany figures, in which:

FIGS. 1A through 1D are images and schematics showing characterizations of fluid loading on a chip (FLO-Chip). FIG. 1A shows a schematic of the microfluidic platform used in fluid loading on a chip. FIG. 1B shows a representative photograph of the microfluidic platform used in FLO-Chip. FIG. 1C shows a schematic of beads anchored to the coverslip via a single dsDNA tether and a differential interference contrast microscopy image of beads anchored to the coverslip via a single dsDNA tether, wherein the scale bars are 2 μm. Application of flow causes ˜2.6 μm displacement of the bead center as the result of the drag force applied on the bead. FIG. 1D shows a schematic of the direction of in-plane and off-plane stretching force (F) applied on a tether bead under flow, calibration of F with respect to the inlet perfusion rate (Q) in the 1500 μm and 2500 μm wide microchannel, and force-extension examination of single tethers under varying force. In FIG. 1D (iii), application of F leads to transverse fluctuation of the tethered bead. Further, the tether length (1) is described according to the solid red line. The black arrowheads denote flow direction, and the scale bars are 2 μm. In FIG. 1D (iv) the transverse fluctuation of a total of six beads was monitored under different flow rates. The dashed red line is a calibration line and indicates a linear relationship, in this embodiment, between drag force and perfusion rate. The calibration line has a slope of approximately 1.36±0.18 pN·s·mm⁻¹.

FIGS. 2A(i) through 2B(iii) are images and schematics of digoxigenin (DIG) and anti-DIG binding interaction. FIG. 2A(i) shows a schematic of the setup used to examine the dissociation force of digoxigenin (DIG) and anti-DIG binding interaction. The tether includes a DIG end that enables binding to beads functionalized with Anti-DIG and a biotin end that enables binding of the tether to the streptavidin on the coverslip surface. FIG. 2A (ii) shows a cumulative probability histogram of the recorded rupture forced under three different loading rates. FIG. 2A (iii) shows the most probable rupture force (F*) with respect to applied loading rate (\dot{F}) for DIG-AntiDIG binding interaction. FIG. 2B (i) schematic of the setup used to execute hydrodynamic force spectroscopy on biotin-streptavidin binding interaction. The tether included two biotin ends that enable binding of the tether to beads functionalized with streptavidin and to the streptavidin on the coverslip surface.

FIG. 2B (ii) shows the cumulative probability histogram of the recorded rupture forces under three different loading rates. FIG. 2B (iii) shows the most probable rupture force with respect to the applied loading rate for biotin-streptavidin binding interaction. For both binding interactions tested in this embodiment, the recorded rupture forces increased with an increase in the applied \dot{F} under semilogarithmic scale. The linear fit, which is represented by the black dash lines, indicated ΔX of 0.8 nm and 1.0 nm along with k_{off} of 4.8×10⁻⁴ s⁻¹ and 7.0×10⁻⁶ s⁻¹ for DIG-AntiDIG and biotin-streptavidin binding interactions, respectively. Furthermore, the extracted energy landscape coefficients for biotin-streptavidin were used to project the most probable rupture force with respect to loading rate for a single biotin-streptavidin binding interaction, which is represented by the green dash line.

FIGS. 3A through 3C are images and schematics of mechanical unzipping of dsDNA. FIG. 3A shows a schematic of the setup used for mechanical unzipping of dsDNA. The DNA tether features a Biotin end that enables anchoring of the tether to the coverslip surface and a single-stranded end that binds to the short oligo, represented by a green oligo. A fraction of the green oligo enables binding to the tether, and another fraction enables binding to another short oligo conjugated to DIG, represented by the orange oligo. Similarly, a fraction of the orange oligo enables binding to the green oligo, and another fraction enables binding to a third short oligo, represented by the red oligo. FIG. 3B shows a cumulative probability histogram of DNA unzipping force for 18 nt dsDNA with 33%, 58%, and 83% GC content. Increase in GC content caused the rupture events to occur at higher forces. FIG. 3C shows a cumulative probability histogram of DNA unzipping events for 9 nt dsDNA with 78% GC content under (i) varying loading rates, and (ii) varying Mg²⁺ concentration in the buffer. In this embodiment, rupture events occurred at higher forces with an increase in loading rate and Mg²⁺ concentration. The most probable rupturing force increased linearly with loading rate under semilogarithmic scale. Performing a linear fit indicated ΔX of 6.9 nm along with k_{off} of 4.9×10⁻⁶ s⁻¹ for the 9 nt DNA unzipping interaction.

FIGS. 4A through 4E are images and schematics of the FLO-Chip microchannels. FIG. 4A shows a schematic of FLO-Chip outline depicting serially connected microchannels with varying width. FIG. 4B (i) shows a representative bright-field image of the FLO-Chip microchannels coated with beads tethered to the coverslip using DIG-AntiDIG binding interaction, the detected bead survival probability, and the bead area. The microchannels in this embodiment are 250 μm, 500 μm, 750 μm and 1000 μm in width and are coated with beads tethered to the coverslip using DIG-AntiDIG binding interactions. The zoomed-in images depict beads that are tethered to the surface via a single tether. The difference between the detected bead center position under backward flow, represented by the red solid line, and forward flow, represented by the cyan solid line, was used to select tethered beads. FIG. 4B (ii) shows the rupture flow rate for each selected bead as determined by monitoring the bead area when the beads were subjected to linearly increasing flow rate. The flow rate at which each bead was undetected was marked as the corresponding rupture flow rate. FIG. 4B (iii) shows the probability of the selected beads to remain bound under increasing flow rate. In this embodiment, the bead rupture was more probable under smaller flow rates in narrower channels. FIG. 4C shows the calibration of bead drag force (F) with respect to the inlet average velocity (V) in the 1000 μm wide microchannel. The calibration line (red dash line) indicated a linear relationship between drag force and average perfusion velocity with slope of approximately 3.01±0.16 pN·s·mm⁻¹. FIG. 4D shows the dynamic force spectroscopy of 9 nt DNA unzipping with 89% GC content under four different loading rates tested simultaneously on the same chip. Figure E (i) shows the dynamic force spectroscopy of DIG-AntiDIG dissociation. Figure E (ii) shows the dynamic force spectroscopy of the 9 nt DNA unzipping tested under a wide range of loading rates (10⁻²-10² pN·s⁻¹ and 10⁻²-10¹ pN·s⁻¹, respectively).

FIG. 5 shows monitoring of bead displacement under different flow conditions and extraction of tether to coverslip anchoring location. The black arrowheads represent flow conditions, and the black cross represents anchoring location. To extract the location at which the tether is anchored to the coverslip, the tethered beads were subjected to 100 μL/min flow rate in each direction. The tether to coverslip anchoring location was extracted by locating the midpoint of the line connecting the bead center, represented by the red solid and dash lines, when subjected to flow conditions 1 and 2. Next, the beads were subjected to flow condition 3: a flow rate of 0.5 μL for 2 minutes. The distance (d) between the average bead center, represented by a black dash line, and the tether to coverslip anchoring location, represented by the black solid line, was used to extract the tether end-to-end distance (l). Moreover, the transverse displacement of the bead center (Y coordinate bead displacement) was used along with tether end-to-end distance to estimate the stretching force according to the equipartition theorem.

FIG. 6 shows monitoring the bead transverse displacement when subjected to different levels of flow rates. In this embodiment, an increase in flow rate led to an increase in stretching force (F) and caused an increase in bead displacement in the direction of the flow (X-direction). Further, an increase in the stretching force resulted in less transverse fluctuation of the bead center (Y-direction) as anticipated based on the equipartition theorem. The black arrowheads denote flow direction.

FIG. 7 shows calibration of stretching force when beads are subjected to different flow rates performed in two of the microchannels. In this embodiment, width of the channels was 1500 μm and 2500 μm.

FIG. 8 shows an approximation of the cumulative probability density function. Including more terms in the series brings the approximate solution closer to the numerical solution. The first 50 terms, represented by the green line, were used to fit the cumulative probability density function to the force spectroscopy data.

FIGS. 9A through 9F are images and schematics of DNA unzipping interactions in a flow cell. FIG. 9A shows a schematic of the flow cell. FIG. 9B shows a representative photo of the flow cell. FIG. 9C shows a schematic and image of tethered beads stretched under flow. FIG. 9D shows a calibration of the flow cell estimating the stretching force under controlled inlet flow rate.

FIG. 9E shows a schematic of the procedure enabling incorporation of DNA unzipping interactions in the flow cell. FIG. 9F shows a force spectroscopy characterization of unzipping of 18 nt double stranded DNA with different GC content.

FIG. 10 shows a schematic of the DNA Origami Nanoplatform (DONS) depicting the location of overhangs and attachment between two complementary DONS and gel electrophoresis and transmission electron microscopy of DOTS confirming its successful synthesis and dimerization, wherein the scale is 50 nm.

FIGS. 11A and 11B are images and schematic of DNA unzipping in DOTS. FIG. 11A shows a schematic of the setup to characterize DNA unzipping in DOTS. FIG. 11B shows 18 nt DNA unzipping force in DOTS on three different locations for 33 GC and 83 GC.

FIG. 12 shows a force readout of DNA unzipping in DOTS comparing two connections versus one connection. Doubling the number of connections caused an increase in rupture force. Moreover, the level of increase in rupture force was dependent on the location at which the connections where incorporated.

FIG. 13 shows a schematic of a FLO-Chip 100 having an inlet port 101 and an outlet port 102 and channels 103 connected by channel connectors 104.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein. Prior to describing the various aspects of the present disclosure, the following definitions are provided and should be used unless otherwise indicated. Additional terms may be defined elsewhere in the present disclosure.

### General Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

“Fluid” as used herein is given its ordinary meaning, i.e., a liquid or a gas. In some embodiments, a fluid is a liquid. The fluid may have any suitable viscosity that permits flow. If two or more fluids are present, each fluid may be independently selected among essentially any fluids (liquids, gases, and the like) by those of ordinary skill in the art, by considering the relationship between the fluids.

“Functionally attached” as used herein refers to a juxtaposition of two or more components arranged such that both components function normally and allow the possibility that at least one of the components can mediate a function that is exerted upon at least one of the other components.

“Microfluidic,” as used herein, refers to a system or device having fluidic conduits or chambers that are generally fabricated at the micron to submicron scale. The microfluidic system of the invention is fabricated from materials to that are compatible with the conditions present in the particular experiment of interest. Such conditions include, but are not limited to, pH, temperature, ionic concentration, pressure, and application of electrical fields. The materials of the device are also chosen for their inertness to components of the experiment to be carried out in the device. Such materials include, but are not limited to, glass, quartz, silicon, and polymeric substrates, e.g., plastics, depending on the intended application.

A “microscale cavity” is a conduit or chamber having at least one dimension between about 0.1 and 500 microns.

A “microchannel” is a channel having at least one microscale dimension, as noted above. A microchannel optionally connects one or more additional structure for moving or containing fluidic or semi-fluidic (e.g., gel- or polymer solution-entrapped) components.

An “ordered array of a plurality of sets of particles” is an array of particle sets (each particle set is constituted of similar or identical particle “members” or “types”) having a spatial arrangement. The spatial arrangement of particle sets can be selected or random. In a preferred embodiment, the spatial arrangement is selected. The arrangement can be known or unknown. In a preferred embodiment, the spatial arrangement of particle sets is known.

A “set” of particles is a group or “packet” of particles having similar or identical constituents.

A “particle movement region” is a region of a microscale element in which particles are moved. A “fluid movement region” is a region of a microscale element in which fluidic components are moved.

A “microwell plate” is a substrate comprising a plurality of regions which retain one or more fluidic components.

Two components are “physically associated” when they are in direct or indirect contact.

As used herein, the term “particles” includes, but is not limited to, nucleic acids, proteins, peptides, compounds, cells (e.g., blood platelets, white blood cells, tumorous cells, embryonic cells, spermatozoa, etc.), synthetic beads (e.g., polystyrene), organelles, and multi-cellular organisms. Particles may include liposomes, proteoliposomes, yeast, bacteria, viruses, pollens, algae, or the like. Particles may also refer to non-biological particles. For example, particles may include metals, minerals, polymeric substances, glasses, ceramics, composites, or the like. Additionally, particles may include cells, genetic material, RNA, DNA, fragments, proteins, etc. or bead, for example, with fluorochrome conjugated antibodies.

As used herein, the term “microfluidic system” refers to a system or device including at least one fluidic channel having microscale dimensions. The microfluidic system may be configured to handle, process, detect, analyze, eject, and/or sort a fluid sample and/or particles within a fluid sample.

The term “channel” as used herein refers to a pathway formed in or through a medium that allows for movement of fluids, such as liquids and gases. The term “micro channel” refers to a channel, preferably formed in a microfluidic system or device. One of ordinary skill in the art will be able to determine an appropriate volume and length of the micro channel for the desired application. The micro channel may have any selected cross-sectional shape or arrangement, non-limiting examples of which include a linear or non-linear configuration, a U-shaped or D-shaped configuration, and/or a rectangular, triangular, elliptical/oval, circular, square, or trapezoidal geometry. A microfluidic device or microfluidic chip may include any suitable number of micro channels for transporting fluids. A microfluidic chip may be provided as a disposable cartridge with a closed channel system.

As used herein the terms “vertical,” “lateral,” “top,” “bottom,” “above”, “below,” “up,” “down,” and other similar phrases should be understood as descriptive terms providing general relationship between depicted features in the figures and not limiting on the claims, especially relating to flow channels and microfluidic chips described herein, which may be operated in any orientation.

### General

Various molecular binding interactions were determined between microbeads on one side and double stranded DNA (dsDNA) tethers on the other side. The dsDNA tethers were anchored to a chemically functionalized glass coverslip and the microbead using specific binding interactions. Thus, this setup enables utilization of hydrodynamic force applied on the microbead to mechanically manipulate a molecular complex or binding interaction. The molecular interactions were examined based on detecting a rupture event due to the hydrodynamic force applied on the microbead. Detection of the rupture event occurs based on removal of the bead, which is a large object that can be imaged. Hence, this approach can be carried out using bright-field imaging on mostly widely available microscopes that would likely already be contained in labs interested in these measurements.

Furthermore, the microfluidic device consists of multiple channels with varying width that are serially connected. This design enables execution of multiple force spectroscopy tests per chip resulting in enhanced throughput. For example, the microfluidic device can be fabricated using SU-8 photolithography followed by polydimethylsiloxane soft lithography to prepare the flow cells. Fabrication of the flow cells is significantly lower in cost than conventional force spectroscopy techniques. Moreover, the strategy implemented here makes single molecule force spectroscopy available to most laboratories with access to basic facilities such as a syringe pump and bright-field microscopy.

This force spectroscopy platform was used to examine some of the previously studied molecular interactions. The force spectroscopy data produced using this microfluidic platform were in close agreement with values reported using previous, more complex force spectroscopy techniques verifying the validity of the findings. Therefore, using microfluidics and hydrodynamic force spectroscopy a low-cost and high-throughput technique has been established that is compatible with setup of most laboratories enabling controlled manipulation of different molecular constructs. Furthermore, the fact that this is carried out on a microfluidic chip means the system is portable and could be coupled with other microfluidic technologies for sorting, purification, and chemistry, for example.

Additional advances include a high throughput aspect to enable simultaneous testing of thousands of interactions, simultaneous testing of different loading rates, like rate of increasing the force) which influences the rupture and simplifying data analysis. Also disclosed is the use of lower cost imaging approaches such as cell phone microscopy that make the system portable and readily available.

### Device

The present disclosure provides for a microfluidic device for measuring hydrodynamic force between two components, wherein the device includes (1) a rigid material, wherein said rigid material comprises at least two channels of varying width that are serially connected; and (2) a first component, wherein said first component is functionally attached to a second component, and further wherein the second component is anchored to the rigid material.

“Channel” is used herein to mean a feature or article that at least partially directs the flow of a fluid. The channel may have any cross-sectional shape (circular, oval, triangular, irregular, square, or rectangular, for example) and in some embodiments, the channel may include characteristics that facilitate control over fluid transport (e.g., structural characteristics) and/or physical or chemical characteristics or other characteristics that can exert a force (e.g., a containing force) on a fluid. In some embodiments, the fluid within the channel may partially fill the channel and in other embodiments, the fluid may completely fill the channel.

The channel may be of any size, including but not limited to a dimension perpendicular to fluid flow from 20 to 2500 microns, 150 to 400 microns, 401 to 650 microns, 651 to 800 microns, or 801 to 1200 microns. In some embodiments, the dimensions of the channel may be chosen such that fluid is able to freely flow through the article or substrate. In other embodiments, the dimensions of the channel may be chosen to allow a certain volumetric or linear flowrate of fluid in the channel. In further embodiments, the number of channels and the shape of the channels can be varied by any method known to those of ordinary skill in the art. There are at least two channels, and in some embodiment, more than two channels may be used. In other embodiment, two or more channels may be used where they are positioned adjacent to each other or parallel to each other.

The term “anchored” includes but is not limited to directly attached or coupled to, or indirectly attached or coupled to an element such as one or more intermediary structures.

In some embodiments, each channel may allow for fluid flow through a proximal end of the channel to a distal end of the channel. In other embodiments, the channels may be connected via at least one channel connector. In further embodiments, the channel connector may allow for fluid flow through a proximal end of the channel connector to a distal end of the channel connector.

The term “channel connector” is used herein to refer to a feature that directs the flow of a fluid between channels. In further embodiments, the channel connectors direct the flow of a fluid from an inlet port, through the channels, and to and out an outlet port.

In some embodiments, the device may comprise an inlet port and an outlet port. In other embodiments, the inlet port may be connected to a proximal end of a channel, and the outlet port may be connected to a distal end of another channel. In further embodiments, the inlet and/or the outlet may be indirectly connected to a channel via a channel connector.

In some embodiments, the rigid material may be a coverslip. “Coverslip” is used herein in accordance with the meaning normally assigned there to in the art and further described below. In some embodiments, the coverslip may be glass. In further embodiments, the glass can be coated and can be printed on.

In other embodiments, the coverslip may be functionalized with polyethylene glycol, biotin-polyethylene glycol, or a combination thereof. In other embodiments, alternatives to polyethylene glycol may be used, including but not limited to polypropylene glycol, polyaminoacids, polyacrylamides, polyvinylpyrrolidone, zwitterionic polymers, and polysaccharides.

In some embodiments, the channels may be made of polydimethylsiloxane (PDMS). In other embodiments, alternatives to PDMS may be used, including but not limited to polystyrene, poly (methyl methacrylate), poly (lactic-co-glycolic acid), thermoplastic elastomer, or Flexdym™ (manufactured by Eden Tech).

In some embodiments, the first component may be a microbead. “Microbead” is used herein in accordance with the meaning normally assigned thereto in the art and further described below. In some embodiments, microbeads may be diameters from 20 nm to 1 mm. In further embodiments, the microbeads may be made from a variety of materials, including but not limited to silica and a variety of polymers, copolymers, and terpolymers.

In some embodiments, the second component may be a first nucleic acid. In other embodiments, the first nucleic acid may be single stranded, and may be affixed to the coverslip at a proximal end. In further embodiments, the first nucleic acid may hybridize with a second nucleic acid at a distal end, thereby forming a double stranded nucleic acid molecule. in some embodiments, the second nucleic acid may be tethered to the microbead on its distal end.

In some embodiments, a nucleic acid may be naturally occurring and in further embodiments may be derived from a biological sample. Nucleic acid may also include nucleic acids with modified backbones. Nucleic acid can be of any length. Nucleic acid may perform any function, known or unknown. The following are non-limiting examples of nucleic acids: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, primers, mitochondrial DNA, cell-free nucleic acids, viral nucleic acid, bacterial nucleic acid, and genomic DNA. A nucleic acid may comprise one or more modified nucleotides, such as methylated nucleotides or methylated nucleotide analogs. If present, modifications to the nucleotide structure may be imparted before or after assembly of the polymer. The sequence of nucleotides may be interrupted by non-nucleotide components. A nucleic acid may be further modified after polymerization, such as by conjugation with a labeling component. A nucleic acid may be single-stranded, double-stranded or have higher numbers strands (e.g., triple-stranded).

The nucleic acid tether can be 500, 1000, 1500, 2000, 2500, 3000, 3500, 4000, 4500, 5000, 5500, 6000, 6500, 7000, or 7500 nucleotides in length, or any amount above, below, or in between. In a specific example, the nucleic acid is 5700 nucleotides long.

The term DNA may be used herein in accordance with the meaning normally assigned thereto in the art and further described below. In some embodiments, DNA may include, but is not limited to, cDNA, plasmids, vectors, isolated DNA of any sequence, isolated RNA of any sequence, nucleic acid probes, primers, and mitochondrial DNA.

The term RNA may be used herein in accordance with the meaning normally assigned thereto in the art and further described below. In further embodiments, RNA may include, but is not limited to, messenger RNA (mRNA), transfer RNA (tRNA), ribosomal RNA (rRNA), short interfering RNA (siRNA), short-hairpin RNA (shRNA), and micro-RNA (miRNA).

In some embodiments, nucleic acid may include synthetic nucleotides, wherein synthetic nucleotide is used herein to refer to a nucleotide that is not known or naturally occurring in nature.

In other embodiments, aptamer may be used to refer to a nucleic acid that has a specific binding affinity for a target molecule. In further embodiments, aptamer may include a set of copies of one type or species of nucleic acid molecule that has a particular nucleotide sequence. An aptamer may include any suitable number of nucleotides and may refer to more than one such set of molecules. Different aptamers may have either the same or different numbers of nucleotides.

In some embodiments, the term tethered may be used herein to mean attached, anchored, or coupled by the use of a tether.

In some embodiments, the first nucleic acid may be affixed to the coverslip via biotin/streptavidin. In other embodiments, the second nucleic acid may be affixed to the microbead via biotin/streptavidin. In further embodiments, the second nucleic acid may be affixed to the microbead via an antibody/antigen interaction. In certain embodiments, the antibody/antigen interaction may be an anti-digoxigenin (Anti-DIG)/digoxigenin (DIG) interaction.

In some embodiments, the first component may be a first protein. In other embodiments, the protein may be affixed to the coverslip at a proximal end. In further embodiments, the protein may be affixed to the coverslip via biotin/streptavidin. The term “protein” is used herein in accordance with the meaning normally assigned thereto in the art and further described below. In some embodiments, protein may include, for example, biotin, streptavidin, avidin, or tamavidin. In some embodiments, the second component may be a microbead. In other embodiments, a second protein may be affixed to the microbead. In further embodiments, the second protein may be affixed to the microbead via biotin/streptavidin.

In some embodiments, the rigid material may include at least 3 channels. In other embodiments, the rigid material may include at least 4 channels. In further embodiments, the rigid material may include at least 5 channels. Also contemplated are 6, 7, 8, 9, or more channels. In some embodiments, each channel may have a different width. In some embodiments, at least one channel may have a width from 20 to 2500 microns. In other embodiments, at least one channel may have a width from 150 to 400 microns. In further embodiments, at least one channel may have a width from 401 to 650 microns. In other embodiments, at least one channel may have a width from 651 to 800 microns. Further, at least one channel may have a width from 801 to 1200 microns.

In some embodiments, the channels may have a height from 2 to 150 microns. In some embodiments, the channels are parallel to each other in the rigid material and create a continuous pathway between the inlet port and the outlet port. In further embodiments, fluid may be forced through the channels. Further, fluid may also be forced through the channel connectors.

In some embodiments, a molecular interaction may occur between the first component and the second component. “Molecular interaction” as used herein means attractive or repellant forces between molecules and between non-bonded atoms. In some embodiments, molecular interactions may include, but are not limited to, hydrophobic interactions, hydrogen bonding, x-stacking, weak hydrogen bonding, amine stacking, electrostatic interactions, Van der Waals forces, antibody-antigen interactions, or cation-I.

### Systems

The present disclosure provides for a system for measuring molecular interactions between molecules, wherein the system includes a microfluidic device comprising a rigid material, wherein said rigid material comprises at least two channels of varying width that are serially connected; and a first component, wherein said first component is functionally attached to a second component, and further wherein the second component is anchored to the rigid material, wherein fluid can be forced through the channels, and further wherein hydrodynamic force between the first component and the second component can be measured.

In some embodiments, the system may further include a detector for measuring dissociation force between the first and second component. In some embodiments, the detector includes a radiation detector that may be configured to detect radiation from the sample flow. Further, an excitation source may be configured to induce the radiation within the sample flow. In other embodiments, other excitation sources may be used, including but not limited to other lasers (e.g., diode lasers), light emitting diodes, and the like. In some embodiments, a plurality of radiation sources such as lasers, and associated detectors may be used. Further, one or more detectors may be configured to receive detected radiation, which may comprise transmitted, scattered and/or fluorescent radiation.

In other embodiments, the detector may include detection of molecules and/or other fluorophores, including single-molecule fluorescence. In further embodiments, the detector may include a flow cytometer, a fluorescence spectrometer, or a laser spectrometer, and may be multi-functional devices having one or more of such functions, and/or other functions. In other embodiments, detectors such as semiconductor optical sensors, including but not limited to photodiodes, may be used. Video and/or imaging sensors may also be included for some applications if desired. The particles may then be characterized within the output channel by optical, electrical, magnetic, ultrasound, or other techniques or combinations thereof.

In some embodiments, the system further comprises a computer for housing the software. In some embodiments, an electronic circuit, including but not limited to a computer, may be used to analyze detector signals, so as to determine properties of the particles. For example, cell dimensions and other properties may be determined, and particles may be imaged, reacted, or otherwise processed. In further embodiments, the detector may include a high-throughput cell cytometer, single-molecule fluorescent spectrometer, genetic analyzer, or fluorescence-activated cell sorter. In further embodiments, particles having detected properties may be counted, extracted, sorted, or otherwise processed.

In some embodiments, the detector can make measurements based on bright-field imaging. In bright-field imaging, illumination light is transmitted through the sample and the contrast is generated by the absorption of light in dense areas of the specimen. Colloidal gold nanoparticles can serve as labels in bright-field microscopy due to their large absorption and scattering cross sections. Software can be used to analyze dissociation force.

In some embodiments, software may be used to analyze dissociation force. In some embodiments, the system may further comprise a computer for housing the software. In further embodiments, the computer may include a minicomputer, a microcomputer, a UNIX® machine, mainframe machine, personal computer (PC) such as INTEL®, APPLE®, or SUN® based processing computer or clone thereof, laptop computer, notebook computer, tablet computer, personal digital assistant (PDA), cellular phone with wide area network access capability, MP3 player or other portable entertainment device having wide area network access capability, or other network-enabled portable digital devices, or other appropriate computer, such as home appliances, televisions, stereos, audio and/or video equipment, including recording devices, security devices, printers, fax machines, other office equipment, medical devices, vehicles cameras, GPS equipment, laboratory equipment, RFID equipment, manufacturing machinery, and other devices having an embedded processor capable of wide area network access, or any machine having or locally associated with storage capability, and having the ability to access a wide area network to function with the present invention. In further embodiments, computer may include any type of network of computers, including but not limited to a network of computers in a business, the Internet, or personal data assistant (PDA).

Embodiments may further include a variety of hardware and/or software elements capable of sending data requests to- and receiving responses from an application server. In some embodiments, the computer may run an operating system including but not limited to variations of the Linux, Unix, Microsoft Disk Operating System (“MS-DOS”), Microsoft Windows, Palm OS, Symbian, Android OS, Apple Mac OS, and/or Apple IOS operating systems. In further embodiments, the computer may also be coupled with a display.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual variability as well as the variability inherent between individuals.

For convenience, the systems of the subject invention may be provided in kits. The kits could include the cells to be used, which may be frozen, refrigerated or treated in some other manner to maintain viability, reagents for measuring the parameters, and software for preparing the screening results. The software will receive the results and perform analysis and can include reference data. The software can also normalize the results with the results from a control culture. The composition may optionally be packaged in a suitable container with written instructions for a desired purpose, such as screening methods, and the like.

For further elaboration of general techniques useful in the practice of this invention, the practitioner can refer to standard textbooks and reviews in cell biology, tissue culture, embryology, and cardiophysiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

### Methods of Using

The present disclosure provides for a method of determining strength of molecular interactions between a first molecule and a second molecule including providing a device, wherein the device comprises (1) a rigid material, wherein said rigid material comprises at least two channels of varying width that are serially connected; (2) a first component with the first molecule associated therewith; and (3) a second component, with a second molecule associated therewith, wherein the first component and the second component can associate with each other through the first and second molecules; executing at least one force spectroscopy test; and measuring dissociation force between the first and the second molecule, thereby determining the strength of the molecular interaction between the first and the second molecule.

The term “force spectroscopy” is used herein to refer to the set of techniques for the study of the interactions and the binding forces between individual molecules. In some embodiments, force spectroscopy may be used to measure the mechanical properties of single polymer molecules or proteins, or individual chemical bonds. The term “dissociation force” is used herein to refer to the force required to separate or split molecules into other elements including, but not limited to, atoms, ions, or radicals.

In some embodiments, each force spectroscopy test is executed under different loading conditions. “Loading conditions” is used herein to refer to conditions under which load is applied to the first and second molecules for purposes of a force spectroscopy test, wherein the conditions may include but are not limited to force applied and rate of applied force. In other embodiments, each force spectroscopy test is executed on different molecular interactions including but not limited to interactions between different proteins and antibodies.

In some embodiments, the rupture force that is most probable under each loading rate is determined from a cumulative distribution of rupture forces based on a rupture model. In further embodiments, the rupture model may include but is not limited to the Evans model or Ritchie model.

In some embodiments, the molecular interaction comprises binding affinity. “Binding affinity” is used herein to refer to the strength of the binding interaction between members of a binding pair.

In other embodiments, the force spectroscopy tests are used to measure the binding affinity of first and second molecule.

In some embodiments, the method may further include detecting the first and second component with bright field microscopes or cellphone-based mobile microscopy.

In some embodiments, “bright-field microscope” is used herein to refer to the device in which illumination light is transmitted through a sample and a contrast is generated by light absorption in dense areas of the specimen, thus allowing a user to closely examine the sample. In some embodiments the bright-field microscope may include, for example, a Nikon Eclipse Ti-E microscope.

In some embodiments, “cellphone-based mobile microscopy” is used herein to refer to the use of a camera-enabled mobile device to perform light microscopy. In some embodiments, cellphone-based mobile microscopy may be used for in-field or point-of-care application for detection of changes in solution rheological properties, altered ion concentration, or the presence or interactions of biomarkers.

In further embodiments, the method may be used for identifying drug candidates. In some embodiments, identifying drug candidates may include drug discovery. “Drug discovery” is used herein to refer to the process of identifying chemical entities that have the potential to be of value as medications. In other embodiments, identifying drug candidates may include drug analysis. “Drug analysis” is used herein to refer to the aspects of identifying novel drugs, assessing their affinity and specificity, characterizing their molecular structures, and testing their efficiency in vitro and in vivo.

In other embodiments, the method may be used for analyzing strength of antigen binding. “Antigen binding” is used herein in accordance with the meaning normally assigned thereto in the art and further described below. In some embodiments, antigen binding may include the bonds between an antigen and antigen-binding molecule caused by hydrogen bonding, electrostatic forces, Van der Waals forces, or some combination thereof.

In other embodiments, the method may be used for screening biological samples. “Screening biological samples” is used herein to refer to the screening of biological samples including, but not limited to, serum, plasma, urine, and blood, for example, for purposes that may include toxicology and diagnostics.

In further embodiments, the method may be used for biopharmaceutical analysis. “Biopharmaceutical analysis” is used herein to refer to the separation and analytical techniques used to identify promising drug targets and candidates with high therapeutic potential.

### Methods of Manufacturing

The present disclosure provides for a method of manufacturing a device, wherein the method includes designing a network of at least two channels; producing transparencies; using the transparencies as masks in photolithography to spin-coat the network of at least two channels onto a wafer to create a microfluidic platform with at least two channels; placing at least one post on the design of at least two channels to form at least one reservoir; casting a prepolymer onto the microfluidic platform; oxidizing the prepolymer in a plasma discharge; and attaching the prepolymer to a rigid material.

In some embodiments, designing the network may be carried out using a CAD program. “CAD Program” is used herein to refer to computer-aided design software used to digitally create 2D drawings and 3D models. In further embodiments, CAD programs include, but are not limited to, AutoCAD, SolidWorks, Inventor, Siemens NX, SelfCAD, TinkerCAD, or Freehand 7.0.

In other embodiments, the curing may take place at about 60-70° C. In further embodiments, the curing may take place for 30 minutes to 2 hours. In some embodiments, the rigid material may be a coverslip.

In some embodiments, the method may further include pegylating the coverslip with polyethylene glycol and biotin-polyethylene glycol, wherein pegylating the coverslip may include immersing the coverslip in a cleaning solution; rinsing the coverslip with water; drying the coverslip; immersing the coverslip in acetone and adding an aminosilane to the acetone; leaving the coverslip in acetone and aminosilane for a reaction time; immersing the coverslip in a mixture of acetone and water; drying the coverslip; preparing a pegylation solution of mPEG and biotin-mPEG; incubating the coverslip with the pegylation solution for a pegylation time; rinsing with water; and drying with N2.

In some embodiments, the cleaning solution is a mixture of H₂O₂ and H₂SO₄. In other embodiments, the cleaning solution may include, but is not limited to, potassium hydroxide/ethanol, NOCHROMIX® (manufactured by Avantor), or NanoStrip® (manufactured by Avantor).

In other embodiments, the aminosilane is (3-aminopropyl) triethoxysilane (APTES). In further embodiments, the aminosilane may include, but is not limited to, 3-aminopropyldimethylethoxysilane (APDMES), 3-aminopropyltrimethoxysilane (APTMS), propyldimethylmethoxysilane (PDMMS), or N-(6-aminohexyl) aminomethyltriethoxysilane.

In some embodiments, the reaction time may be from 1 to 5 minutes. In other embodiments, the pegylation time may be from 60 to 120 minutes.

In some embodiments, the channels may be casted in PDMS. In other embodiments, the channels may be casted in alternatives to PDMS as discussed above. In further embodiments, the method may further comprise constructing a tether. In some embodiments, “tether” is used to mean a feature that anchors the second component to the rigid material or to a second set of molecules that is attached to the rigid material. In some embodiments the tether may include a nucleic acid, or more specifically, DNA, RNA, synthetic nucleotide, or any combination thereof.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Low-Cost and Massively Parallel Force Spectroscopy with FLO-Chip

### Introduction

Herein, a low-cost, high throughput technique is demonstrated using microfluidics for multiplexed application of molecular fluid loading on-a-chip (FLO-Chip). The FLO-Chip design includes multiple serially connected microchannels with varying width, enabling multiple flow-rupture tests and multiplexed mechanical manipulation of up to approximately 4000 data points per chip. By using low-cost microfluidic chip fabrication, standard sample preparation, and conventional widefield microscopy, the cost for each test experiment may be substantially lower using this methodology compared to other cost-efficient force spectroscopy techniques. Single molecule force spectroscopy measurements may be demonstrated by dissociating Biotin-Streptavidin and DIG-Anti DIG binding interactions along with unzipping of double stranded DNA of varying sequence and under different dynamic loading rates and solution conditions. The FLO-Chip enables rapid testing under varying loading rates and ion concentrations, which highlights its versatility for biophysical studies of molecular mechanobiology.

This study presents a low-cost, high throughput technique using microfluidics for massively parallel application of molecular fluid loading on-a-chip (FLO-Chip) to test various molecular interactions under a wide range of mechanical loads (approximately 0.1 pN-100 pN) and loading rates (10⁻²-10² pNs⁻¹). Utilizing fluid flow to apply mechanical tension on molecular interactions is implementable without the need for specialized instrumentation. (Oijen, et al.; Elshenawy et al.; Kim et al.) Moreover, the approach outlined herein utilizes polydimethylsiloxane (PDMS)-based soft lithography to enable production of minimal cost flow cells (approximately $3 per chip) bonded on chemically functionalized coverslips. (McDonald et al.) The implemented flow cells consist of multiple microchannels with different widths that are serially connected. Since the flow velocity is a function of the channel geometry, having channels with different widths enables execution of multiple irreversible single molecule bond rupture tests under varying loading conditions (e.g. loading rates) on one chip resulting in significantly enhanced experimental throughput (up to approximately 4000 measurements per chip within approximately 2 hours of total experiment time). In addition, these tests can be evaluated using standard imaging equipment that is widely available to most biology, biophysics, biomaterials and bioengineering laboratories, thereby eliminating the need for specialized force spectroscopy instrumentation and broadly increasing access to single molecule force studies.

For verification, extension of double stranded DNA (dsDNA) tethers under controlled tension were studied and the previously reported worm-like chain behavior was reproduced.

Furthermore, dissociation of biotin-streptavidin and digoxigenin (DIG)-AntiDIG (digoxigenin antibody) was examined along with unzipping of double stranded DNA with different stem length and sequences under various dynamic loading rates and solution conditions. These experiments yielded most probable rupture forces and binding interaction free energy landscape parameters that agreed well with prior results using more complex force spectroscopy techniques. Therefore, the approach enables a high-throughput single molecule force spectroscopy on-a-chip with low-cost and a preparation procedure that can be integrated into a wide range of laboratories for rapid physical characterization of molecular complexes.

### Results

### 2.1 FLO-Chip Design and Force Calibration

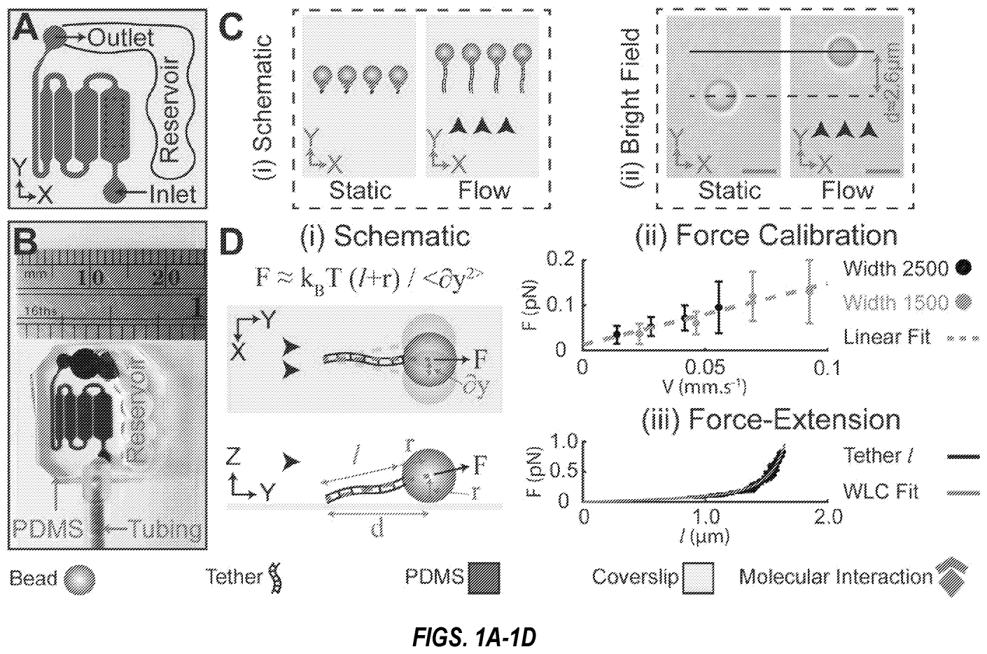

The throughput of force spectroscopy is often limited by one-at-a-time loading as in optical tweezers or atomic force microscopy (AFM) measurements. Methodologies such as magnetic tweezers or centrifugal force microscopy allow multiplexed application of mechanical loading^{[2b]}, but they remain limited to what interactions can be tracked in a single field of view because all interactions in the assay (including ones that are not being monitored) experience the same loads. Here this limitation was overcome in multiplexing through a unique microfluidic chip design that consists of multiple, serially connected microchannels with varying width, allowing for loading some samples to rupture while limiting loads in other channels. This enabled execution of multiple flow rupture tests per chip resulting in significantly enhanced throughput. FIG. 1 illustrates the design approach in FLO-Chip with multiple microchannels of varying width (500 μm, 1000 μm, 1500 μm, 2000 μm, and 2500 μm) and fixed height (120 μm) that were serially connected. The local drag force scales with flow velocity, and the flow velocity is inversely proportional to the width of the channel for a constant channel height, which is the case here. Hence, for a given volumetric flow rate, the narrowest channel exhibits the largest force followed by the channels of increasing width that experience ½, ⅓, ¼, and ⅕ the force and loading rate, respectively. This design was chosen to enable multiple sets of force spectroscopy measurements per chip, either under similar (i.e. repeats) or distinct loading conditions, resulting in significantly enhanced throughput (FIG. 1A, B).

To enable utilization of fluid flow to mechanically manipulate binding interactions, the molecular construct of interest was sandwiched between a microbead (2.2 μm average diameter) on one side and a 5700 bp double stranded DNA (dsDNA) tether anchored to a glass coverslip on the other side. The dsDNA tether was anchored to a glass surface by biotin-streptavidin binding. Subjecting the anchored samples to fluid flow causes a drag force on the microbeads that stretches the dsDNA tethers and translates the drag force to the molecule or interaction of interest (FIG. 1C). The parabolic profile of flow near the coverslip surface results in a lift force on the anchored microbeads, which minimizes non-specific interactions between the bead and the coverslip surface (FIG. 1D (i)). (Zhang et al.)

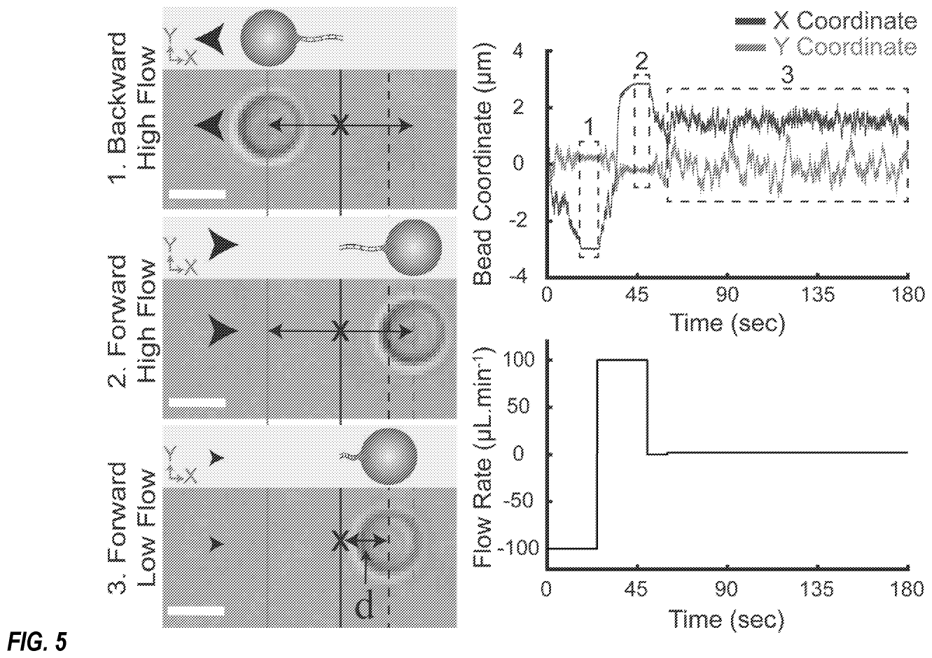

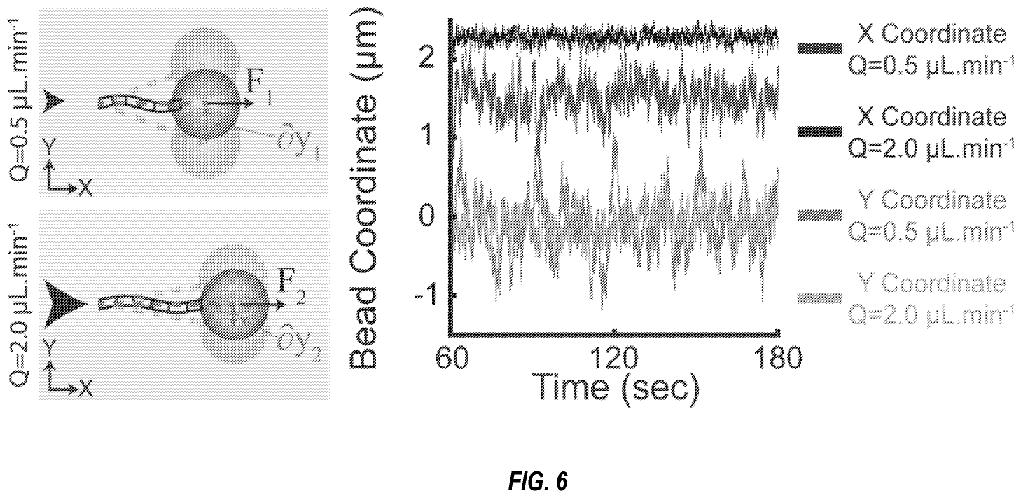

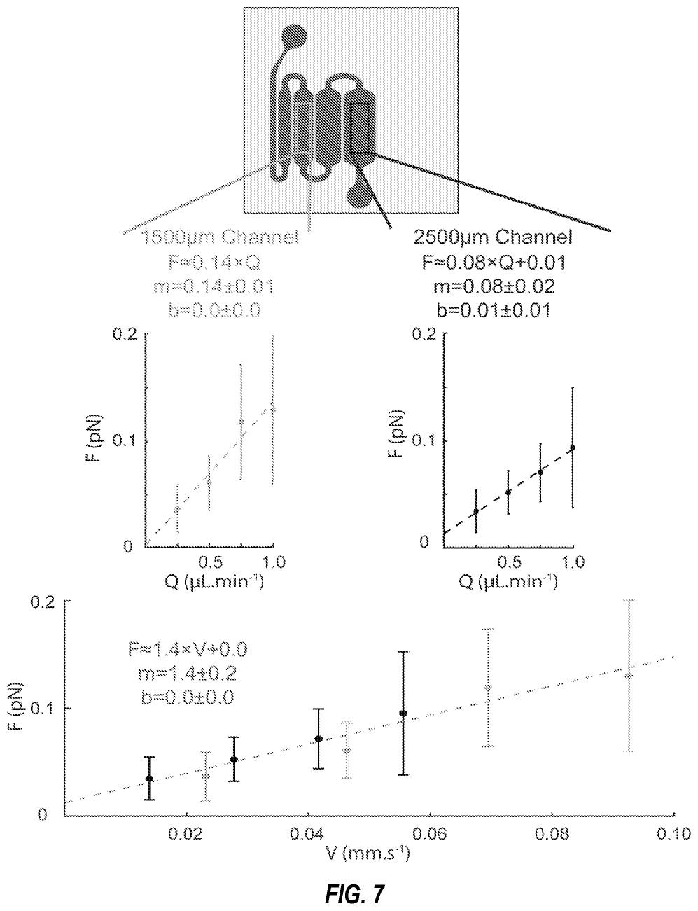

The input parameter in FLO-Chip is the perfusion rate, Q (i.e. volumetric flow rate) controlled using a syringe pump. However, to use FLO-Chip for single molecule force spectroscopy, the level of drag force that is applied on tethered microbeads (F) when subjected to perfusion needed to be calibrated. Therefore, as a first step, the level of drag force (F) was estimated and applied on the beads with respect to the perfusion rate (Q). To this end, the tethered beads were subjected to a range of perfusion rates and monitored the mean-square displacement of the bead center in the transverse direction by video monitoring (FIG. 1D (i); FIG. 5). The level of F with respect to Q was calibrated based on the equipartition theorem according to equation:

$$F = \frac{{k}_{B} T ( l + r )}{< δ {y}^{2} >} \qquad ( 1 )$$

where k_{B} is the Boltzmann constant, Tis the absolute temperature, I is the end-to-end length of the DNA tether, r is the bead radius, and <δy²> is the variance of bead center displacement in the direction transverse to the flow (FIG. 1D(i), (ii); FIG. 6). (Strick et al. 1996; Strick et al. 1998) This approach has been previously implemented to estimate the tether stretching force in hydrodynamic (Kim et al.) and magnetic tweezer (Le et al.) based force spectroscopy. Calibration was performed in two of the microchannels (1500 μm and 2500 μm in width) by monitoring displacement of up to 30 beads per microchannel when subjected to four different perfusion flow rates (FIG. 7). As anticipated, F increased linearly with respect to Q in each channel. The slope of the linear fit was used as the calibration coefficient to determine F for any given Q. Moreover, the slope extracted from each linear fit decreased proportionally with the microchannel width which was anticipated as the average flow velocity decreases proportionally to the width of each channel for any given flow rate (FIG. 7). Therefore, the values were calibrated for F estimated based on equation 1 with respect to the average flow velocity in each of the two calibrated channels (FIG. 1D (iii); FIG. 7). The linear fit revealed a slope of 1.36±0.18 pN·s·mm⁻¹ that was used to estimate F in each FLO-Chip microchannel. It is worth noting that the performed calibration is only valid for beads of the same diameter (2.2 μm in average), tether length (approximately 1.9 μm), and microchannel height (120 μm). The FLO-Chip must be recalibrated if any of these factors change when performing single molecule force spectroscopy experiments.

2.2 Force-Extension Behavior of dsDNA

To verify the estimated F with respect to average perfusion velocity, FLO-Chip was used to study the extension behavior of the dsDNA tether under force. Beads were subjected that were anchored to the coverslip via a single tether to a force ramp and monitored the increase in/upon the increasing F (FIG. 1D (i), (iii)). The average force-extension curve was well described by the worm-like chain (WLC) model (Petrosyan et al.) for dsDNA (persistence length was 56±8 nm, contour length was 1.8±0.1 μm, total of 9 beads analyzed). The extracted average persistence length and contour length were in good agreement with values reported for dsDNA persistence length (Bustamante et al.) and the anticipated contour length value for the 5700 nt the tether (1.9 μm if 0.34 nm length per base pair for dsDNA is assumed). (Mandelkern et al.) These results verify the validity of using the force versus volumetric flow rate calibration to estimate F, and demonstrate the capability to study the force-extension behavior of biopolymers.

### 2.3 Rupture Forces of Protein-Small Molecule Interactions

A key goal of the FLO-Chip system is to enable testing of forces required to rupture molecular interactions. The FLO-Chip assay requires at least two molecular interactions: one to anchor one of the dsDNA tether ends to the coverslip surface, and another to attach the other end of the tether to the microbead. In principle, since the tether stretching force is applied on both interactions simultaneously, FLO-Chip can be used to examine the rupture force required to dissociate either interaction. However, this would require synthesis of a new tether for every interaction of interest. To enable a versatile setup that can incorporate various molecular interactions for rupture force measurements, DNA base-pairing was used to bind the interaction of interest onto the tether. In addition, the assay requires stable binding to the surface. Hence, a dsDNA tether was prepared with a biotin at one end, for surface attachment, and a 30 nt ssDNA overhang at the other end, for attaching the interaction of interest. Based on previous studies, it was estimated biotin-streptavidin would withstand force up to approximately 40 pN, and a 30 bp DNA duplex (in a shearing configuration) would withstand forces up to 42-55 pN at the loading rates of interest (approximately 0.1-10 pN/s). (Strunz et al.) Hence, this setup could be used to probe interactions with rupture forces up to approximately 40 pN over a range of loading rates approximately 0.1-10 pN/s.

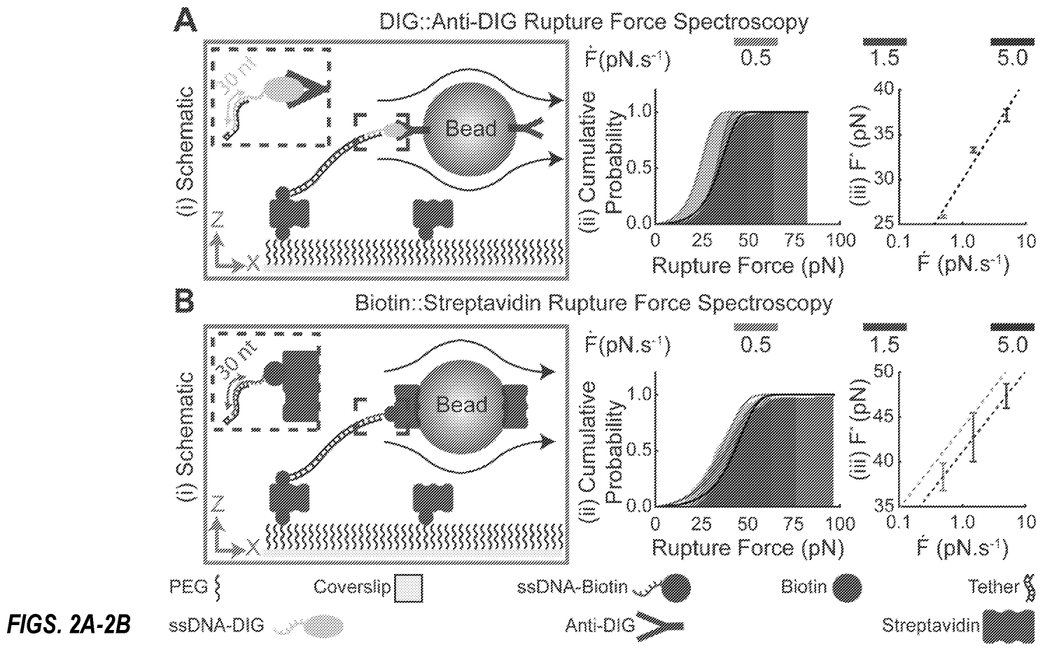

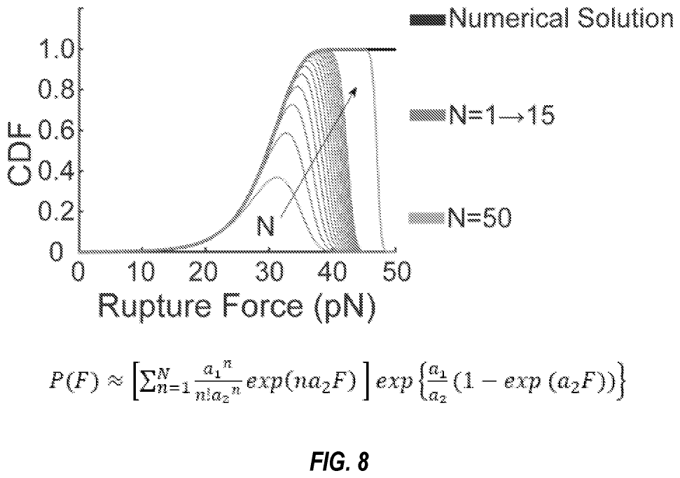

As proof of concept, well-characterized interactions were studied, starting with digoxigenin (Dig) binding with an anti-Digoxigenin antibody (AntiDig), which was expected to rupture at forces of approximately 20 pN within loading rate range of approximately 0.1-10 pN/s. (Strick et al. 1996; Neuert et al.) To this end, a ssDNA was annealed with DIG with complementary sequence to the free end of the tether to enable binding to microbeads coated with AntiDIG (FIG. 2A (i)). The strength of DIG-AntiDIG binding interactions was tested under three different loading rates (Fequal to 0.5, 1.5 and 5.0 pN/s) (FIG. 2A (ii), (iii)). A statistical distribution of rupture events was observed, which shifted to higher forces with increasing loading rate, as illustrated in FIG. 2A(ii) in terms of cumulative distribution functions. The most probable rupture force (F*) under each loading rate was determined from the cumulative distribution of rupture forces based on the Evans and Ritchie rupture model. (Evans et al.) (FIG. 8) Rupture forces for DIG-AntiDIG of F*=25.9±0.2 pN, 33.2±0.3 pN and 37.2±0.7 pN was measured under loading rates of 0.5 pNs⁻¹, 1.5 pNs⁻¹ and 5.0 pNs-1, respectively, which agrees well with previous studies using atomic force microscopy (˜30 pN under ˜100 pN/s loading rate) (Neuert et al.) and acoustic force spectroscopy (˜26 pN under ˜2 pN/s loading rate). (Sitters et al.) According to Evans and Ritchie, (^{[19]} the most

probable rupture force F* can be defined as:

$${F}^{*} = \frac{{k}_{B} T}{Δ X} ln \frac{F Δ X}{{k}_{B} T \cdot {k}_{off}} \qquad ( 2 )$$

where k_{off} is the dissociation rate at zero force, ΔX is the distance to the transition state and \dot{F} is the applied loading rate. The extracted F* as a function of loading rate (FIG. 2A(iii)) was fit to the Evans and Ritchie model (Equation 2) to determine k_{off}=4.4×10⁻⁴ s⁻¹ and ΔX=0.8 nm for the DIG-AntiDIG binding interaction, which are in the range of previously reported values. (Strick et al. 1996; Neuert et al.)

As a next step, Biotin-Streptavidin, another well-characterized interaction, was tested. (Merkel et al.) In this case, single stranded DNA oligos modified with Biotin were annealed to the ssDNA end of the tether along with using beads coated with Streptavidin. (FIG. 2B (i)) Similar to DIG-AntiDIG, the dissociation force for biotin-streptavidin binding is lower than the level of rupture force for DNA unbinding in shear. (Merkel et al.; Wang et al. 2013) Therefore, it was anticipated the rupture to occur due to dissociation of the biotin-streptavidin interaction near the bead or the coverslip surface. It is worth noting that both tether ends could possibly bind to the glass coverslip preventing anchoring of the streptavidin-coated beads. As implemented previously by Yang et al., (Kim et al.) limiting the number of available streptavidin covering the coverslip surface can enable tethers that are only bound to the coverslip surface on one end. However, significant reduction in experimental throughput (approximately 400 data points per chip) occurred when tethers with Biotin on both ends were utilized. This yield could be increased by varying the ratio between PEG and Biotin-PEG on the glass coverslip, but the throughput was reasonable for this analysis. Biotin-streptavidin binding was tested under three different loading rates and a similar shift was observed to larger rupture forces with increasing loading rate (FIG. 2B (ii), (iii)). Similar to DIG-AntiDIG, the F* values for biotin-streptavidin under each loading rate were extracted from the cumulative distributions of rupture forces. Since two identical biotin-streptavidin interactions are connected serially under this configuration, bond dissociation under mechanical load ideally is two times more likely to occur compared to a single biotin-streptavidin binding interaction which results in slightly weakened attachment. According to Evans, (Evans et al. 2001) the most probable rupture force for N serially connected binding interactions can theoretically be described as:

$${F}^{*} = \frac{{k}_{B} T}{Δ X} ln \frac{F Δ X}{N \cdot {k}_{B} T \cdot {k}_{off}} \qquad ( 3 )$$

Patel et al. previously showed equation 3 accurately describes the rupture force of two iminobiotin-streptavidin binding interactions that were serially connected compared to the corresponding rupture force for a single iminobiotin-streptavidin interaction in AFM measurements. (Patel et al.) Therefore, the F* values obtained for biotin-streptavidin with respect to loading rate using FLO-Chip were fit with the modified Evans model (Equation 3) revealing k_{off}=7.0×10⁻⁶ s⁻¹ and ΔX=1.0 nm. These values were used to determine the rupture force a single binding interaction according to Equation 2. The projected values for F* with respect to loading rate along with the extracted values for k_{off} were in the range of previously reported values for biotin-streptavidin binding, while the extracted value of ΔX=1.0 nm is slightly higher compared to previously reported values (approximately 0.5 nm). (Merkel et al.; Yuan et al.) The observed discrepancy in AIX may bel due to role of cooperative unbinding of multiple, serially connected interactions in increasing the apparent distance to transition state as described by Evans. (Evans et al. 2001)

2.4 Rupture Forces for Unzipping of dsDNA

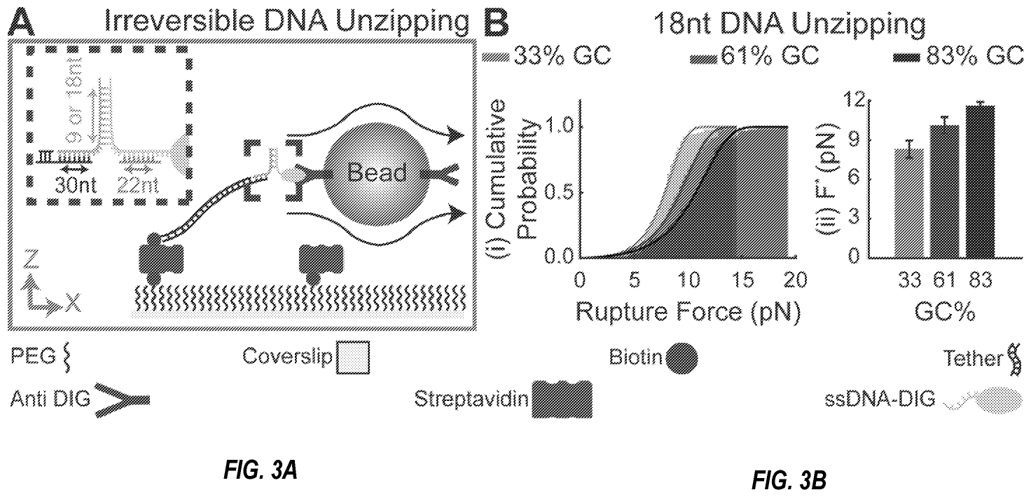

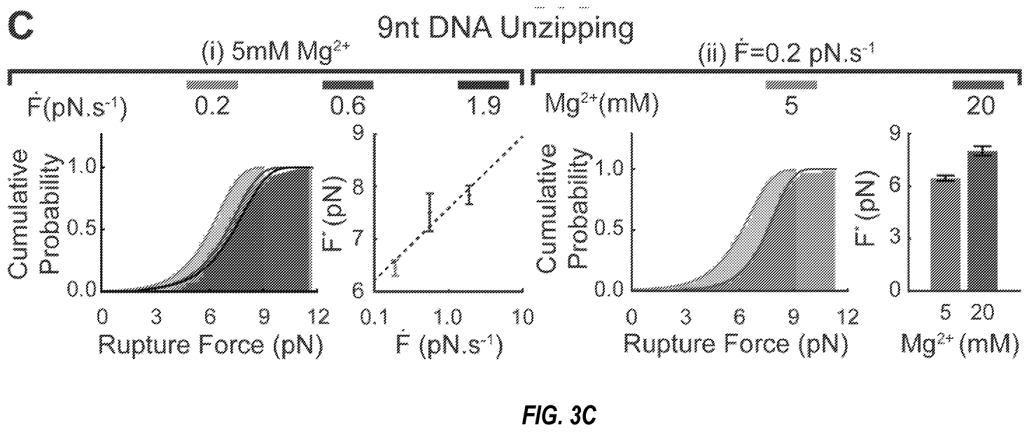

Binding between two complementary ssDNA strands has recently been implemented to develop a variety of molecular mechanical probes. (Ma et al.) FLO-Chip was used to quantify the rupture force required to unzip these dsDNA mechanical probe interactions. To this end, a ssDNA oligonucleotide was used with the sequence of interest to the single stranded end of the tether that contained the complementary sequence followed by an additional sequence to attach to a complementary strand modified with DIG (FIG. 3A). This yielded an assay with the desired DNA binding interaction (in an unzipping configuration) sandwiched between the tether and a bead coated with Anti-DIG (FIG. 3A). A third oligo (red in FIG. 3A inset) was also annealed that was complementary to a fraction of the DIG-conjugated oligo to fortify the distance between the dsDNA unzipping interaction and the AntiDIG coated bead. First the role of GC content was examined in regulating the force required to unzip an 18 bp long dsDNA duplex of varying sequence. To enable direct comparison of the FLO-Chip force spectroscopy data with previous studies, the same 18 bp dsDNA duplex sequence with 83% GC content was used that was previously used as a cellular tension sensor. (Wang et al.) The interactions were subjected to a 0.19 pNs⁻¹ loading rate in 5 mM Mg²⁺ and observed an increase in rupture force with increase in GC content (FIG. 3B(i)), which is consistent with prior studies. (Woodside et al.; Zhang et al.) Fitting the Evans and Ritchie model (Evans et al.,) to each cumulative probability rupture force distribution revealed an increase in the most probable rupture force with increasing GC content (F*=8.3±0.6 pN, 10.2±0.6 pN and 11.6±0.3 pN for 33%, 61% and 83% GC content, respectively) (FIG. 3B(ii)). Previous studies revealed a force of 12-14 pN for the 18 bp DNA duplex with 83% GC content, (Wang et al.; Mosayebi et al.) which is in close agreement with the recorded value of F*=11.6 pN. It is worth noting that the reported 12-14 pN rupture force reported for the 18 bp dsDNA duplex with 83% GC content is the rupture force under which 50% of the interactions rupture in less than 2 seconds under constant mechanical load, whereas the current studies allow for direct quantification of the most probable rupture force at a given loading rate.

Next, FLO-Chip was used to probe the rupture force for a 9 bp dsDNA duplex sequence that was also previously used in the design of hairpin-based cellular tension probes. (Zhang et al.; Dutta et al.) The interaction (78% GC) was subjected to various loading rates and increasing loading rates were observed, resulted in higher unzipping forces (FIG. 3C(i)). The results revealed most probable rupture forces for the 9 nt DNA unzipping interaction of F*=6.5±0.2 pN, 7.5±0.4 pN and 7.8±0.2 pN under loading rates of 0.2 pNs⁻¹, 0.6 pNs⁻¹ and 1.9 pNs-1, respectively. Previous measurements using biomembrane probe force spectroscopy on unzipping of a DNA hairpin with a stem sequence identical to the 9 nt unzipping interaction tested at 500 pNs⁻¹ revealed F* of approximately 9.8±3.4 pN, (Dutta et al.) which is in close agreement with the projected value of approximately 11.3 pN estimated based on the loading rate dependence. (FIG. 3C(i)) The slight discrepancy in rupture force is likely due to the effect of the stem loop in the DNA hairpin. Moreover, the reported 9.8 pN force for unzipping the 9 bp dsDNA hairpin is the value at which the interaction exhibits 50% probability of unzipping, which is theoretically lower than the most probable rupture force reported here. Fitting the most probable rupture force versus loading rate results to the Evans and Ritchie model (Equation 2) revealed a dissociation rate at zero force of k_{off}=4.9×10⁻⁶ s⁻¹ and a distance to transition state of ΔX=6.9 nm for the Int DNA unzipping. These values were in the range of coefficients reported for DNA hairpin unzipping interactions with similar stem length and GC content examined using optical trap force spectroscopy. (Woodside et al.)

An important parameter influencing the stability of DNA binding interactions Is the cation concentration. The effects of increasing Mg²⁺ concentration was tested, which is known to increase DNA melting temperatures. (Owcarzy et al.) Increasing the Mg²⁺ concentration from 5 mM to 20 mM under 0.2 pN·s⁻¹ loading rate caused the F* to increase from 6.5±0.2 pN to 8.0±0.3 pN. These results are consistent with the expected increase in stability and demonstrate the capacity to use the FLO-Chip assay to test interactions under a variety of solution conditions.

### 2.5 Massively Parallel Hydrodynamic Force Spectroscopy Under Multiplexed Fluid Loading Rates

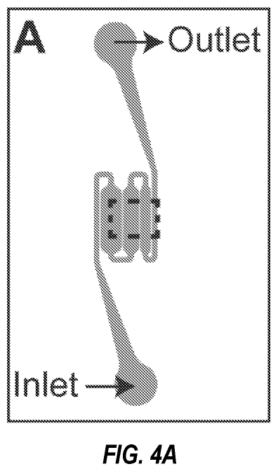

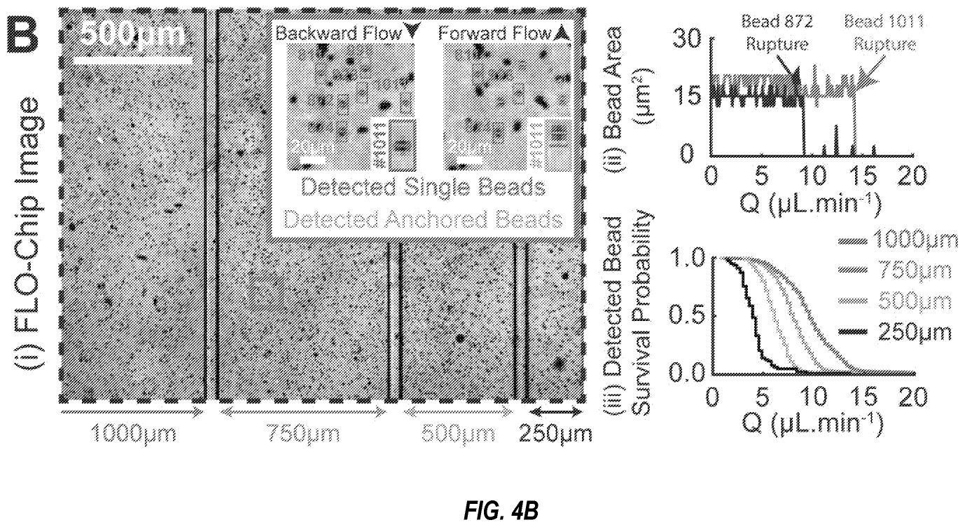

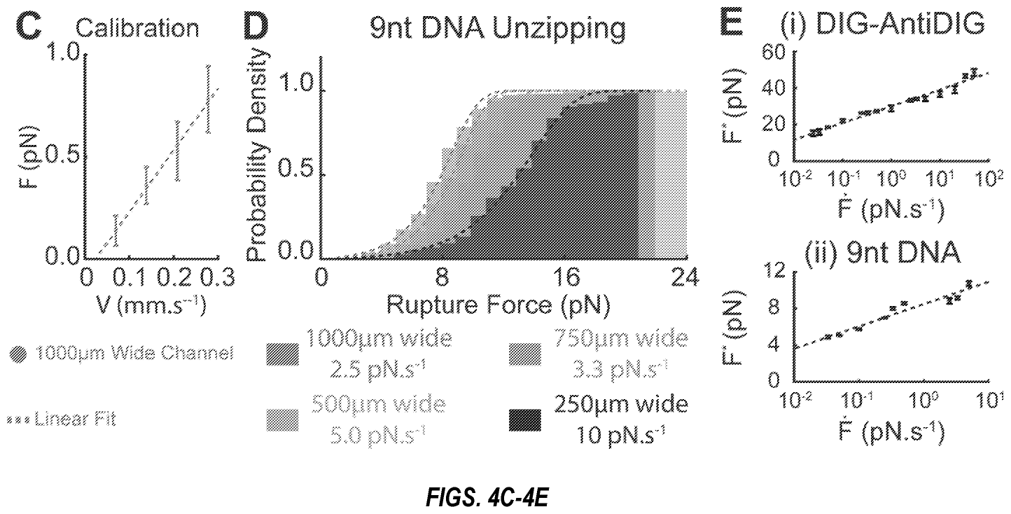

Multiplexed testing of different loading rates on the same chip can significantly enhance the throughput of rupture force characterization under a wide range of dynamic mechanical loads. To enable multiplexing of fluid loading rates in a single experiment, a different version of the FLO-Chip was designed that allows for channels of varying width (250 μm, 500 μm, 750 μm, and 1000 μm) and fixed height (60 μm) to be imaged simultaneously in one field of view (FIG. 4 A, B(i)). To automate identification of beads tethered to the surface via a single DNA tether, the bead positions were monitored when subjected to two opposite fluid flows that cause stretching of the DNA tether in each direction. The beads that exhibited significant movement (greater than approximately 2 μm) when subjected to each fluid flow were selected for force spectroscopy analysis (FIG. 4 B(i)). Furthermore, detection of a rupture event was also automated by monitoring the bead pixel area when subjected to a linearly increasing flow rate. Upon rupture, the bead immediately flows away, and the flow rate when the pixel area drops to zero is marked as the corresponding rupture flow rate (FIG. 4 B(ii)). This automation allows for rapid characterization of the full probability distribution of rupture forces simultaneously in all microchannels, each subjected to a distinct loading rate. Since the average velocity and stretching force increase proportionally with decreasing channel width, bead rupture occurs first in the narrowest channel followed by beads in the channels with increasing widths (FIG. 4 Biii). This automated bead selection and rupture flow rate estimation allowed for highly efficient analysis of the time-lapse images (approximately 5 minutes total analysis time for approximately 4000 beads using MATLAB).

Similar to the previous FLO-Chip design, the level of drag force (F) applied on the beads was determined with respect to the perfusion average velocity (V). Calibration was performed in the widest microchannel (1000 μmin width) by monitoring displacement of approximately 30 beads per microchannel when subjected to four different perfusion flow rates. As anticipated, F increased linearly with respect to V (FIG. 4C). The linear fit revealed a slope of 3.01 pN·s·mm⁻¹, which was used to estimate F in each FLO-Chip microchannel.

Using this chip, dynamic force spectroscopy measurements were repeated on DIG-AntiDIG dissociation (four chips tested for four sets of loading rates in the 10⁻²-10² pNs⁻¹ range, approximately 4000 beads analyzed per chip) and 9 nt DNA unzipping (three chips tested for three sets of loading rates in the 10⁻²-10¹ pNs⁻¹ range, approximately 2000 beads analyzed per chip). Representative cumulative probability rupture force distributions for 9 nt DNA unzipping extracted from a single experiment on one chip illustrates the ability to test four different loading rates simultaneously (FIG. 4D). The resulting F* versus loading rate for DIG-AntiDIG (FIG. 4 E(i)) was fit to the Evans and Ritchie model (Evans et al. 1997) (Equation 2) to determine k_{off}=1.2×10⁻⁴ s⁻¹ and ΔX=1.0 nm for the DIG-AntiDIG binding interaction. Similarly, for the 9 nt DNA unzipping interaction, the F* versus loading rate (FIG. 4 E(ii)) was fit to the Evans and Ritchie model (Evans et al. 1997) (Equation 2) to determine k_{off}=3.1×10⁻⁴ s⁻¹ and ΔX=3.9 nm for the 9 nt DNA unzipping interaction. The estimated values of k_{off} and ΔX for DIG-AntiDIG dissociation and 9 nt DNA unzipping were in close agreement with the corresponding values estimated in FIG. 2A (iii) and FIG. 3C(i).

### Discussion

This study reports on the use of FLO-Chip to characterize to simultaneously characterize up to thousands of molecular binding interactions in parallel. Probing interactions that are widely used in single molecule assays (DIG-Ant-DIG and biotin-streptavidin) and in molecular force sensing experiments (DNA-DNA unzipping) were the focus. For the interactions that have been previously studied, the values for most probable rupture force and energy landscape parameters determined using FLO-Chip agreed well with previously reported values using other force spectroscopy techniques, verifying the validity of the FLO-Chip assay. Hence, FLO-Chip provides similar high-quality force characterization as other single molecule force spectroscopy techniques while offering the significant advantages of reduced cost for each experiment along with execution of massively parallel mechanical manipulation of molecular binding interactions under multiplexed mechanical loading rates.

Furthermore, the multiplexing range enabled using FLO-Chip is significantly enhanced compared to the throughputs reported using magnetic tweezers (Guizar-Sicairos et al.) optical manipulation (Chiou et al.) and on-a-chip atomic force spectroscopy. (Otten et al.) Moreover, the number of readouts per chip presented in this study are approximately 3 times higher than throughputs reported using centrifugal force spectroscopy (Kim et al.) and acoustic force spectroscopy. (Strick et al. 1996; Strick et al. 1997) Furthermore, the capacity to multiplex loading rates by simultaneously imaging multiple channels with varying widths was introduced, thereby enabling full characterization of molecular binding interactions in one test. Finally, the automated bead selection and rupture detection results in significantly reduced total analysis time of the force spectroscopy test images (total analysis time of approximately 5 min using MATLAB programming for total of approximately 4000 analyzed beads). Furthermore, FLO-Chip leverages a preparation procedure prior to each force spectroscopy test (approximately 2 hours). Hence, full mechanical characterization of a molecular interaction can be carried out in a few hours as opposed to several days or even weeks with other methods.

While recent efforts such as centrifugal force spectroscopy also reduce the cost associated with force spectroscopy, (Kim et al.) FLO-Chip can further reduce the cost of mechanical characterization of molecular interactions. The use of PDMS in FLO-Chip enables significant reduction in cost of each device (approximately $3 per chip). Furthermore, FLO-Chip relies of conventional microscopes and syringe pumps resulting in significantly reduced instrumentation cost. Further reduction in total cost can be considered if a lower-cost syringe pump is used to probe the rupture force under constant mechanical load, and the FLO-Chip readout only requires the ability to detect micron-sized beads in bright field imaging, which could be carried out on existing microscopes in many research laboratories. Therefore, FLO-Chip enables single molecule force spectroscopy tests with substantially lowered total cost compared to similar force spectroscopy approaches such as inexpensive optical tweezers (Smith et al.) (up to 90% lowered total cost) and centrifugal force microscopy (Kim et al.) (up to 80% lowered total cost). It is worth noting that one needs to rely on more advanced force spectroscopy techniques such as optical tweezers if there is a need for high spatial (nanometer scale) and temporal (millisecond range) resolution. (Ha et al.; McDonald et al.)

The compact and portable nature of the FLO-Chip system also opens the possibility for performing force spectroscopy in college- or possibly high school-level educational laboratories that contain conventional bright field microscopes. Furthermore, recent advancements in cellphone-based mobile microscopy (Maamari et al.) could provide a route to eliminating the need for a microscope altogether, which could even further reduce barriers for research and educational laboratories. Finally, the prospect of integrating the FLO-Chip assay with cellphone-based microscopy can enable in-field or point-of-care application for detection of changes in solution rheological properties, altered ion concentration, or the presence or interactions of biomarkers. Moreover, the use of microfluidics can enable integration of single molecule force spectroscopy into the vast applications of microfluidics in biomedical and biophysical research. (Otten et al.; Sackmann et al.) Overall, FLO-Chip can make single molecule force spectroscopy a safe tool with lowered cost and increased throughput, suitable for most laboratories.

### Additional Information for Example 1

The coverslip functionalization with biotin-PEG was performed by piranha cleaning coverslips followed by aminosilanization and reaction with PEG which included a small fraction of biotin-PEG as previously reported. (Ha 2008). Briefly, coverslips were immersed in piranha solution for 30 min (20% H₂O₂ (Sigma Aldrich) and 80% concentrated H₂SO₄ (Sigma Aldrich)) followed by thorough rinsing with water (Milli-Q) (3×). To ensure that the coverslip surface is water-free, the coverslips were then dried overnight at 65 C prior to the silane reaction. Prior to aminosilanization, the coverslips were swirled in Acetone (Sigma) for 10 min followed by slow addition of (3-aminopropyl) triethoxysilane to final concentration of 2% vol/vol. After 2 min of silane reaction, the coverslips were immersed in 1:1 vol/vol mixture of acetone and water to quench the silane reaction followed by thorough rinsing with water (Milli-Q) (3×). The coverslips were then dried overnight at 65 C prior to pegylation.

To pegylate the coverslips, a solution containing 10% wt/vol mPEG (5 kDa, Sigma Aldrich) and 0.2% wt/vol Biotin-mPEG (Sigma Aldrich) was prepared in K₂B₄O₇ (Sigma Aldrich) was prepared to enable 50:1 w/w ratio between mPEG and Biotin-mPEG. The silanized coverslips were incubated with the prepared mPEG/Biotin-mPEG solution for 90 min followed by thorough rinsing with water (Milli-Q) (3×) to remove the excess PEG. Next, the coverslips were dried with dry N2 and stored in room temperature.

### Microfluidic Flow Cell Preparation

The microfluidic platform was fabricated using SU-8 photolithography and polydimethylsiloxane (PDMS) soft lithography (McDonald). Briefly, SU-8 2050 was spin-coated on a 4 in Silicon wafer (University Wafers) followed by UV exposure through a transparency mask and development to replicate the designed microfluidic patterns. The Silicon wafer with the fabricated monolithic features (120 μm in height) was then used to cast the microchannels in PDMS. Following PDMS development, individual flow cells were cut and plasma-bonded on the coverslips functionalized with mPEG/Biotin-mPEG to form the flow cell.

Following the formation of the flow cells, the channels were flushed with 0.1 mg/mL Streptavidin (Sigma Aldrich) in PBS (Fisher Scientific) and incubated for 10 min to enable binding between free streptavidin and the Biotin-mPEG on the coverslip surface. The flow cells were flushed with PBS to remove the streptavidin excess. Next, the flow cells were flushed with blocking buffer (1 mg/mL BSA (Life Technologies), 5 mM Tris (Sigma Aldrich), 5 mM NaCl (Sigma Aldrich), 1 mM EDTA (Sigma Aldrich), 3 mM NaN₃ (Sigma Aldrich), 0.1% vol/vol Tween-20 (Sigma Aldrich)) and incubated for 30 to reduce nonspecific interactions with the coverslip surface. Next, the coverslips were flushed with experimental buffer (5 mM Tris (Sigma Aldrich), 5 mM NaCl (Sigma Aldrich), 1 mM EDTA (Sigma Aldrich), 0.1% vol/vol Tween-20, 5 mM MgCl₂ (Sigma Aldrich)) to remove the excess blocking buffer prior to each force spectroscopy experiment.

### Tether Construct Preparation

Tether preparation starts with the digestion of a approximately 2 μm double stranded plasmid by the BsaI restriction enzyme (NEB R0535S) in 1× CutSmart Buffer (NEB B7204S). Depending on the age of the enzyme, 1-3 units of BsaI per μg of plasmid in a 10 μL reaction volume is usually sufficient to cut the plasmid without over-digestion. The incubation is carried out at 37° C. for 60 minutes followed by enzyme inactivation at 65° C. for 20 minutes. This enzyme cuts the plasmid downstream of its recognition site, leaving a four base single stranded overhang. For each end of the tether, a pair of oligos pre-annealed at room temperature for 15 minutes in 50 mM NaCl can be ligated on using T4 DNA Ligase (NEB M0202S) in 1×T4 ligase buffer (NEB B0202S). In order to facilitate ligation, the oligo containing the 5′ end that will ligate to the BsaI cut plasmid end must be kinased using T4 Polynucleotide kinase (PNK) (NEB M0201S). Incubation is carried out with a T4 PNK concentration of 10 Units per 300 pmole ends for 90 minutes at 37° C., then 20 minutes at 65° C. Because the oligos will be subsequently ligated, the reaction is carried out in 1× T4 ligase buffer instead of T4 PNK buffer. One oligo pair facilitates attachment to the slide via a biotin-streptavidin-biotin connection and the other pair contains a 30 nt overhang. The ligation is carried out with a final enzyme reaction concentration of 4 Units/μL, DNA concentration of 200 nM, and 100-fold excess of both pairs of oligo ends (compared to the linearized plasmid). It is incubated for 30 minutes at room temperature followed by a heat shock at 65° C. for 25 min. After the ligation, EDTA is added to make a final concentration of 20 mM in order to chelate excess magnesium in case any ligase survived the heat shock.

Before HPLC purification, the sample must go through a phenol-chloroform extraction to remove any proteins, such as BSA, that have the potential to stick to the HPLC column. HPLC purification is carried out on a Gen-Pak column (Waters WAT015490) with a salt ramp going from TE100 (25 mM Tris-HCL, 1 mM EDTA, 100 mM NaCl) to TE1000 (25 mM Tris-HCL, 1 mM EDTA, 1M NaCl) to remove the excess oligo ends from the final tether product. A small amount of the fractions collected from the HPLC are then run on a 0.7% agarose gel at 225 volts for 35 minutes, then post-stained with Ethidium Bromide. This ensures good separation of the excess oligo ends from the final tether product and confirms what fractions to keep. The remaining amount of the fractions containing the desired final product are then concentrated and buffer exchanged into 0.5× TE for a total of 3 times using a 30K amicon centrifuge filter (Sigma UFC203024). The final concentration of the tether is then determined via the 260 nm absorbance peak on a spectrophotometer.

### Assembly of Beads Tethered to the Coverslip Via a Single DNA Tether

DNA tethers were preincubated at 200 pM with excess end oligos at 45 C for 1 hour followed by incubation at 37° C. for 1 hour prior to each experiment. For the experiments involving dissociation of Biotin-Streptavidin and Digoxigenin-Anti Digoxigenin, the Biotin-ssDNA end oligo was added at 100× excess to the tether. For the experiment involving unzipping of double stranded DNA, the Cy3-ssDNA was added at 100× excess, and the Dig-ssDNA and Cy5-ssDNA were added at 200×.

Following the incubation of tether with end oligos, the flow cells were flushed with the tether solution and incubated for 30 min at room temperature to enable anchoring of the tether to the coverslip through binding between streptavidin on surface and biotin on the tether end. Next, the flow cells were flushed with the experimental buffer to remove the excess tether followed by addition of the beads. The beads were added at 0.1 wt/vol concentration with 10% blocking buffer and 5 mM MgCl₂. The 10% blocking buffer was added to reduce bead aggregation and non-specific interaction between the surface and the beads during the experiments.

### Flow Application

The flow cell was connected to a 2.5 mL Harvard Syringe 1000 (Fisher Scientific) via translucent tubes with 0.8 mm inner diameter (Cole-Parmer). The syringe was connected to a programmable syringe pump (Harvard Apparatus) to apply flow within the microchannels. Prior to start of the experiment, the beads were allowed to bind to the free end of the anchored tethers for 15 minutes under static condition. Next, the excess beads were washed using a small flow rate prior to the start of each experiment. To apply controlled levels of loading rate on the studied molecular interactions, the beads were subjected to a linear flow ramp programmed using the syringe pump. The slope of the ramp was controlled in order to produce a desired loading rate.

### Flow Cell Calibration

The flow cells were calibrated using equipartition theorem as previously described. (Kim et al.) A calibration was performed in the microchannel with largest width (2500 μm in width×120 μm in height), and the obtained calibration chart was interpolated to the microchannels according to the channel width. In order to determine the location of the base of the DNA tether through which each bead is anchored to the coverslip, the bead center was monitored while subjecting the beads to 100 μL/min backward flow followed by 100 μL/min forward flow. These flow rates were chosen to enable full stretching of the 5745 nt dsDNA tethers. Subjection to these flow rates results in equivalent stretching of the DNA tether in each direction. Therefore, the midpoint along the line that connects the bead center position recorded under stretched condition in each direction denotes the location at which the tethers are anchored to the coverslip (FIG. 5). Next, the beads were subjected to a series of flow rates ranging from 0.25 μL/min to 1.0 μL/min. The beads were kept subject to each flow rate for 2 min to record the average mean-squared lateral fluctuation of the center of the bead (<δy²>) along with the length of the stretched tether (l). The beads were imaged at 50 Hz to extract an accurate mean square lateral displacement of the bead center. Moreover, the end-to-end length of the stretched tether (l) was obtained based on averaged displacement of bead center along the flow direction and the bead radius (r) (FIG. 1D (i)). According to the equipartition theorem, the stretching force applied on the tether (F) can be defined as:

$$F = \frac{{k}_{B} T ( l + r )}{< δ {y}^{2} >} \qquad ( 1 )$$

where k_{B} is the Boltzmann constant and T is temperature. (Strick et al. 1996) This equation suggest that the level transverse fluctuations of the bead center decrease when the bead is subjected to higher forces (FIG. 6). The calibration and all the test experiments were performed at room temperature (23° C.) correlating to k_{B}T=4.114 pN·nm.

### Image Acquisition and Analysis

Bright field imaging was performed using a Nikon TiE. Perfect focus was used to maintain the beads in focus during the course of each measurement. For the experiments pertaining calibration, the beads were imaged using a 100× oil immersion objective. All images were analyzed using a Custom-Built MATLAB code. The epifluorescence images of the beads obtained from the time-lapse epifluorescence imaging files were first converted to binary format to detect the location of each bead. Bead displacement was detected with subpixel resolution (10 nm) by making a Fourier transform of the bead image at each time point followed by making cross-correlation of the Fourier transform image with respect to the Fourier transform corresponding to bead image at time zero. The cross-correlated image was then submerged in an expanded matrix with 10 nm resolution. An inverse Fourier Transform was then performed on the submerged matrix. The row and column index corresponding to the overall maxima of the inverse Fourier transform matrix denotes the subpixel in-plane displacement of the bead with respect to time zero. Since performing the inverse Fourier Transform on the larger submerged matrix requires a high computational cost, a previously reported localized submerging strategy was used (Guizar-Sicarios et al.) to significantly reduce the computational cost of the subpixel bead tracking analysis.

For the force spectroscopy experiments, the beads were imaged using a 40× oil immersion objective. The time of each rupturing event was recorded manually when a bead anchored through a tether was detached and flushed away. The recorded time was then used along with the applied loading rate to extract the force at which each molecular interaction ruptures. The beads that did not displace significantly (approximately 2 μm) were detected as non-specific interactions and were excluded from the analysis. For massively parallel force spectroscopy tests under multiplexed mechanical loading rates, the chip was imaged using a 4× air objective on Nikon TS-100. Beads were subjected to two opposite flow rates to stretch the DNA tether in each direction. Beads that displaced significantly (greater than 2 μm) were automatically selected using a custom-built MATLAB code. Rupture flow rate for each bead was estimated by converting the bright-field time lapse images to binary format and monitoring the detected bead area. The flow rate at which each bead is first undetected is marked as the corresponding rupture flow rate using a custom-built MATLAB code.

### Data Analysis

The probability density of rupture p (F) for a molecular receptor-ligand binding interaction for a given force (F) can be described as:

$$p ( F ) = \frac{{k}_{off}}{F} exp ( \frac{F Δ X}{{k}_{B} T} ) exp ( \frac{{k}_{B} T \cdot {k}_{off}}{F Δ X} ( 1 - exp ( \frac{F Δ X}{{k}_{B} T} ) ) \qquad ( 2 )$$

where k_{off} is the dissociation rate at zero force, ΔX is the potential width and \dot{F} is the applied loading rate (Evans 1997). The cumulative probability of rupture occurrence P (F) for a given force (F) can be estimated with:

$$\begin{matrix}P ( F ) = {\int }_{0}^{F} p ( f ) & df \approx \end{matrix} [ {\sum }_{n = 1}^{N} \frac{{a}_{\text{?}}^{n}}{\text{?}} exp ( {na}_{2} F ) ] exp \{ \frac{{a}_{\text{?}}}{{a}_{\text{?}}} ( 1 - exp ( {a}_{2} F ) ) \} \qquad ( 3 )$$

$$where {a}_{1} = \frac{{k}_{off}}{F} and {a}_{2} = \frac{Δ X}{{k}_{B} T} .$$

$$\text{?} \text{indicates text missing or illegible when filed}$$

The obtained rupturing force population for each experimental condition was binned cumulatively to plot cumulative rupture probability histograms with respect to applied force. The cumulative probability histograms were fitted with equation 3 with the corresponding loading rate as an input to extract ΔX and k_{off}. For each fit, the estimated solution in equation 3 including the first 50 terms were used to accurately estimate the cumulative probability density function (FIG. 5). The extracted coefficients k_{off} and ΔX along with the corresponding loading rate \dot{F} were then used to report the most probable rupture force F* according to:

$${F}^{*} = \frac{{k}_{B} T}{Δ X} ln \frac{F Δ X}{{k}_{B} T \cdot {k}_{off}} \qquad ( 4 )$$

(Evans 2001) The extracted F* were plotted in a force versus logarithmic loading rate diagram. Performing linear fit on the F* versus logarithmic loading rate diagrams was used to extract 4× and K_{off} according to equation 4.

For N bonds connected serially, the apparent most probable rupture force F* for uncooperative rupture can be described as:

$${F}^{*} = \frac{{k}_{B} T}{Δ X} ln \frac{F Δ X}{N \cdot {k}_{B} T \cdot {k}_{off}} \qquad ( 5 )$$

Thus, the apparent rupture force is slightly weakened by kgT/ΔX ln N compared to the strength of a single binding interaction.

### Statistical Analysis

The most probable rupturing force for each experimental condition was reported in mean±standard deviation format. To report statistical analysis, the rupturing force population from each experimental condition was randomly divided to 3 subgroups using random selection without replication. Each subgroup population was then binned cumulatively and fitted with Equation 3 using lead-squared curve fitting to report three most probable rupturing force values for each experimental condition. The three obtained force values were then used to report statistically averaged most probable rupturing force values for each experimental condition.

TABLE 1
List of oligos detailing the sequence.

| Oligo | Sequence |
|---|---|
| Tether single stranded end | CGGTCGAGCTATTGAAAGCTAGCTAGTTGTCCTTGT |
| | CTACCTGGGTGCGCACGA (SEQ ID NO: 1) |
| Tether single stranded end | AGCTAGCTTTCAATAGCTCG (SEQ ID NO: 2) |
| complementary | |
| Tether Biotin end | /5BiotinTEG/CCTACACTGGGATAATTGAC |
| | (SEQ ID NO: 3) |
| Tether Biotin end | ACCGGTCAATTATCCCAGTGTAGG (SEQ ID NO: 4) |
| complementary | |
| ssDNA-Biotin | /5BiotinTEG/TTTTTTTCGTGCGCACCCAGGTAGACAA |
| | GGACAACT (SEQ ID NO: 5) |
| SSDNA-DIG | /5DIG/TTTTTTTCGTGCGCACCCAGGTAGACAAGGAC |
| | AACT (SEQ ID NO: 6) |
| 18 nt DNA Unzipping 33% | TATCAACAGTGAACCATATTTTCGTGCGCACCCAGGT |
| GC Green Oligo | AGACAAGGACAACT (SEQ ID NO: 7) |
| 18 nt DNA Unzipping 58% | AGCCAGCAGAGACACACGTTTTCGTGCGCACCCAG |
| GC Green Oligo | GTAGACAAGGACAACT (SEQ ID NO: 8) |
| 18 nt DNA Unzipping 83% | GGCCCGCAGCGACCACCCTTTTCGTGCGCACCCAG |
| GC Green Oligo | GTAGACAAGGACAACT (SEQ ID NO: 9) |
| 18 nt DNA Unzipping 33% | /5DIG/TTTTTTCTCTGGTTAACGTGTCTGGGCATTTTA |
| GC Gold Oligo | TGGTTCACTGTTGATA (SEQ ID NO: 10) |
| 18 nt DNA Unzipping 58% | /5DIG/TTTTTTCTCTGGTTAACGTGTCTGGGCATTTCG |
| GC Gold DIG Oligo | TGTGTCTCTGCTGGCT (SEQ ID NO: 11) |
| 18 nt DNA Unzipping 83% | 5'dig/TTTTTTCTCTGGTTAACGTGTCTGGGCATTTG |
| GC Gold DIG Oligo | GGTGGTCGCTGCGGGCC (SEQ ID NO: 12) |
| 9 nt DNA Unzipping Green | CGCGCGTACTTTTCGTGCGCACCCAGGTAGACAAGG |
| Oligo | ACAACT (SEQ ID NO: 13) |
| 9 nt DNA Unzipping Gold | /5DIG/TTTTTTCTCTGGTTAACGTGTCTGGGCATTTGT |
| DIG Oligo | ACGCGCG (SEQ ID NO: 14) |
| ssDNA-DIG | TGCCCAGACACGTTAACCAGAG |
| Complementary Red Oligo | (SEQ ID NO: 15) |

### Example 2. Multiplexed DNA Origami Tension Sensor (DOTS) with Tunable Sensitivity

DNA nanotechnology has enabled mapping of biomolecular forces (Polacheck et al. 2016). However, there is need for probes with more advanced functionality and readout capability DNA origami the programmable self-assembly of DNA based nanostructures, enables the design of 3 D nano constructs with unique features that can enable design of complex cellular biophysical probes.

### Method

DNA origami nanotechnology was used to design molecular force probes with a wide range of sensitivities. Hydrodynamic force spectroscopy was used to calibrate various DNA origami tension sensor (DOTS) configurations.

### Results

A hydrodynamic force spectroscopy platform was developed using microfluidics to enable calibration of DOTS DNA unzipping interaction was examined when sandwiched between two DNA origami nanostructures forming DOTS. Significant change in DNA unzipping force was observed depending on the location and number of the interactions.

### Conclusions

Use of DNA origami to design cellular force probes can lead to development of next generation of cellular biophysical probes with advanced functionality and readout complexity.

Various methods have been used to map cellular forces (Polacheck et al. 2016). DNA nanotechnology, in particular, has enabled development of molecular probes enabling mapping of cellular biomolecular forces (Wang et al. 2013; Zhang et al. 2014; Dutta et al. 2018). DNA origami, molecular self-assembly of folding a single stranded DNA known as scaffold using smaller DNA strands, allows for designing 3D DNA Origami nanostructures (DONS) that can enable development of complex cellular force probe (Rothemund 2006; Castro et al. 2011; Wang et al. 2017; and Akbari et al. 2017).

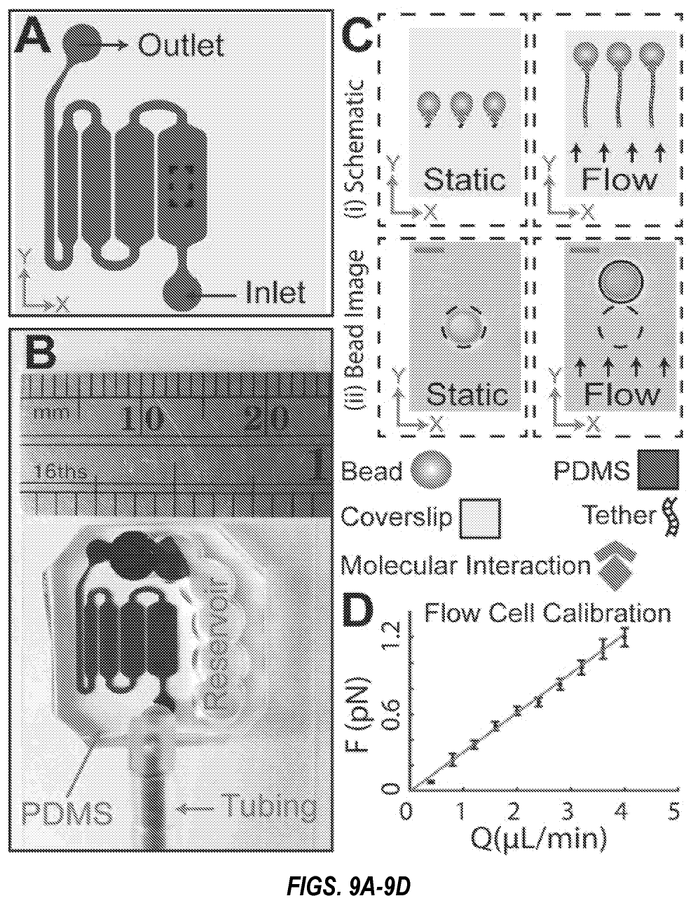

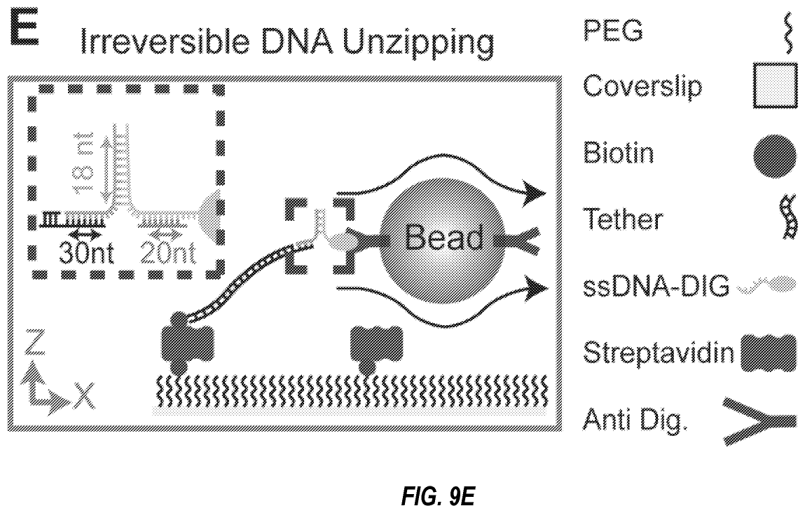

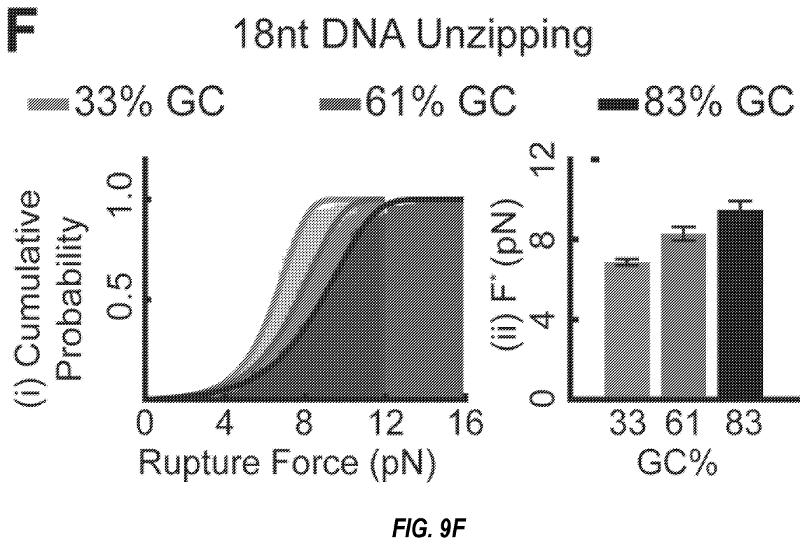

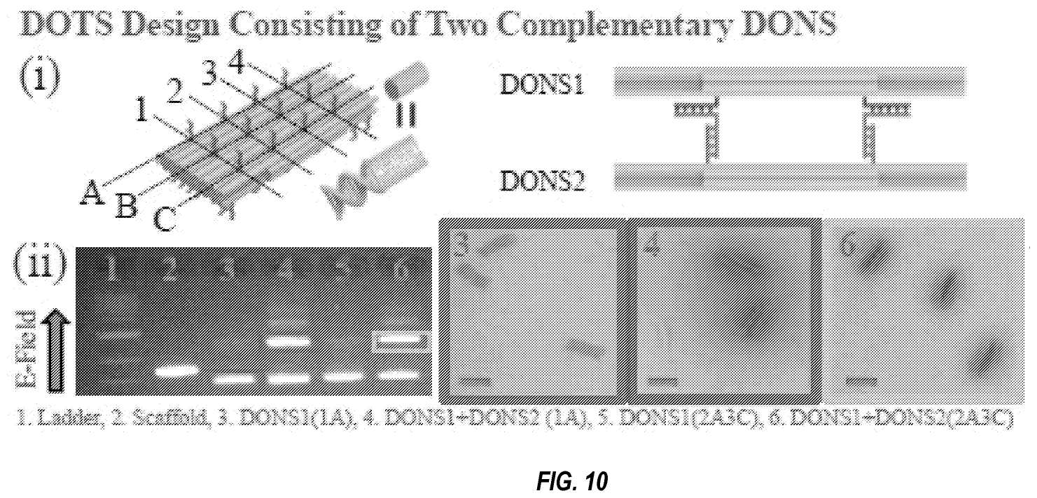

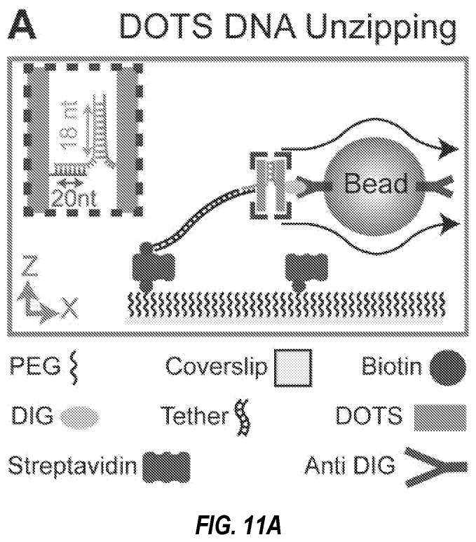

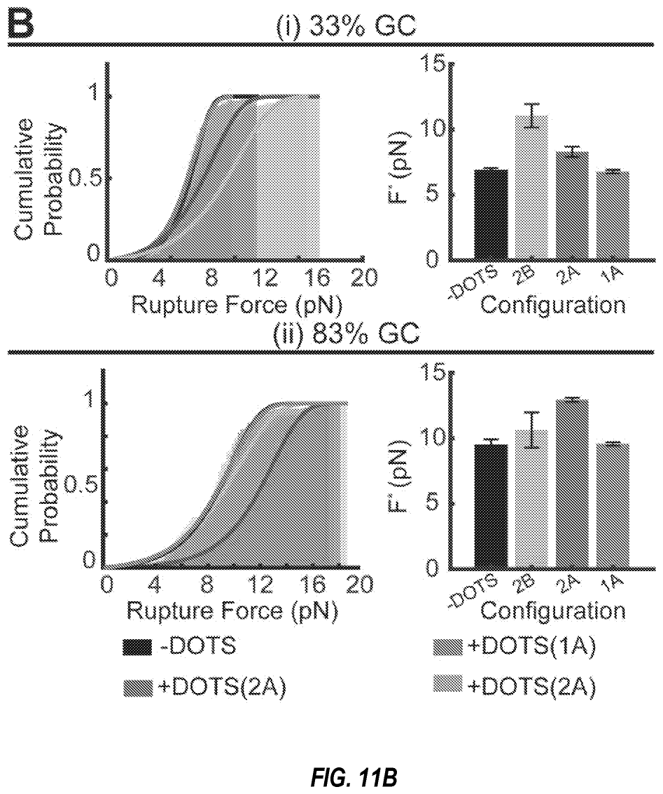

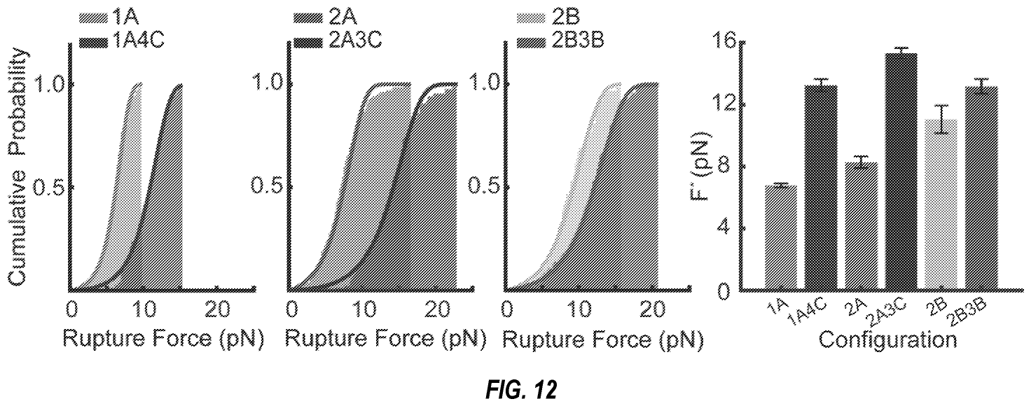

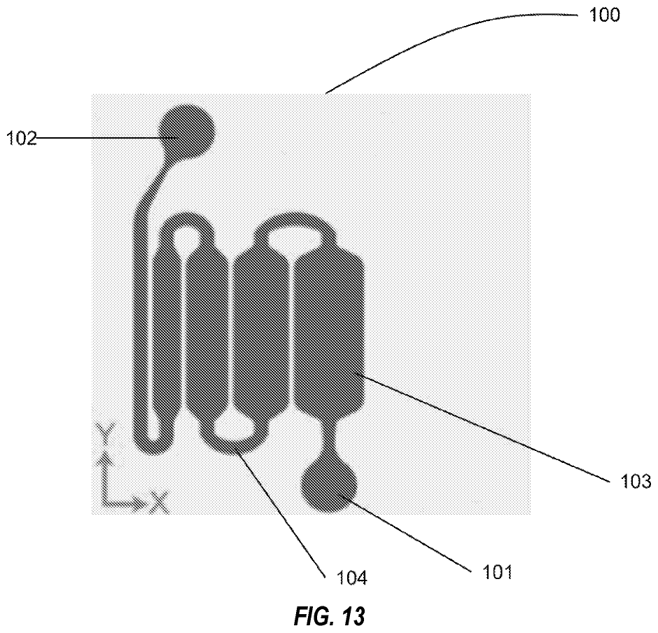

The various designs can be found in FIGS. 9-12.

A low cost, high throughput hydrodynamic force spectroscopy platform was developed to characterize DNA unzipping in DOTS. A highly configurable molecular force probe was developed and characterized using DNA origami. Incorporation of DNA unzipping in DOTS caused significant change in the rupture force depending on the location on the DONS. Increase in number of connections in DOTS caused significant increase in the probe sensitivity depending on the interaction location.

The reported approach on design of highly configurable DOTS along with previous reports on incorporation of DONS on cell membrane (Akbari et al. 2017) enables development of advanced molecular probes that are incorporable on cell membrane to serve as cellular biophysical probes.

Publications cited herein are hereby specifically incorporated by reference in their entireties and at least for the material for which they are cited.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

### Reference List #1

[1] a) T. Ha, I. Rasnik, W. Cheng, H. P. Babcock, G. H. Gauss, T. M. Lohman, S. Chu, Nature 2002, 419, 638; b) R. Roy, S. Hohng, T. Ha, Nat. Methods 2008, 5, 507.
[2] J. C. McDonald, D. C. Duffy, J. R. Anderson, D. T. Chiu, H. Wu, O. J. Schueller, G. M. Whitesides, ELECTROPHORESIS: An International Journal 2000, 21, 27.
[3] S. Kim, P. C. Blainey, C. M. Schroeder, X. S. Xie, Nat. Methods 2007, 4, 397.
[4] a) T. R. Strick, J. F. Allemand, D. Bensimon, A. Bensimon, V. Croquette, Science 1996, 271, 1835; b) T. Strick, J.-F. Allemand, D. Bensimon, V. Croquette, Biophys. J. 1998, 74, 2016.
[5] M. Guizar-Sicairos, S. T. Thurman, J. R. Fienup, Optics letters 2008, 33, 156.
[6] a) E. Evans, K. Ritchie, Biophys. J. 1997, 72, 1541; b) E. Evans, Annual review of biophysics and biomolecular structure 2001, 30, 105.

### Reference List #2

[1] a) W. J. Greenleaf, M. T. Woodside, S. M. Block, Annu Rev Biophys Biomol Struct 2007, 36, 171; b) F. Ritort, J Phys Condens Matter 2006, 18, R531; c) K. M. Herbert, W. J. Greenleaf, S. M. Block, Annu. Rev. Biochem. 2008, 77, 149; d) D. J. Muller, J. Helenius, D. Alsteens, Y. F. Dufrene, Nat. Chem. Biol. 2009, 5, 383; e) E. A. Abbondanzieri, W. J. Greenleaf, J. W. Shaevitz, R. Landick, S. M. Block, Nature 2005, 438, 460; f) C. Bustamante, W. Cheng, Y. X. Mejia, Cell 2011, 144, 480.
[2] a) K. C. Neuman, A. J. N. m. Nagy, 2008, 5, 491; b) B. Nathwani, W. M. Shih, W. P. Wong, Biophys. J. 2018, 115, 2279.
[3] D. Yang, A. Ward, K. Halvorsen, W. P. Wong, Nature communications 2016, 7, 1.
[4] G. Sitters, D. Kamsma, G. Thalhammer, M. Ritsch-Marte, E. J. Peterman, G. J. Wuite, Nat. Methods 2015, 12, 47.
[5] I. De Vlaminck, T. Henighan, M. T. van Loenhout, I. Pfeiffer, J. Huijts, J. W. Kerssemakers, A. J. Katan, A. van Langen-Suurling, E. van der Drift, C. Wyman, C. Dekker, Nano Lett. 2011, 11, 5489.
[6] M. Soltani, J. Lin, R. A. Forties, J. T. Inman, S. N. Saraf, R. M. Fulbright, M. Lipson, M. D. Wang, Nat Nanotechnol 2014, 9, 448.
[7] a) A. M. van Oijen, P. C. Blainey, D. J. Crampton, C. C. Richardson, T. Ellenberger, X. S. Xie, Science 2003, 301, 1235; b) K. E. Duderstadt, H. J. Geertsema, S. A. Stratmann, C. M. Punter, A. W. Kulczyk, C. C. Richardson, A. M. van Oijen, Mol. Cell 2016, 64, 1035; c) S. Kim, P. C. Blainey, C. M. Schroeder, X. S. Xie, Nat. Methods 2007, 4, 397.
[8] a) M. M. Elshenawy, S. Jergic, Z. Q. Xu, M. A. Sobhy, M. Takahashi, A. J. Oakley, N. E. Dixon, S. M. Hamdan, Nature 2015, 525, 394; b) S. Kim, P. C. Blainey, C. M. Schroeder, X. S. Xie, Nat. Methods 2007, 4, 397.
[9] J. C. McDonald, D. C. Duffy, J. R. Anderson, D. T. Chiu, H. Wu, O. J. Schueller, G. M. Whitesides, Electrophoresis 2000, 21, 27.
[10] a) R. Petrosyan, Rheologica Acta 2017, 56, 21; b) J. F. Marko, E. D. Siggia, Macromolecules 1995, 28, 8759.
[11] J. Zhang, S. Yan, D. Yuan, G. Alici, N. T. Nguyen, M. Ebrahimi Warkiani, W. Li, Lab Chip 2016, 16, 10.
[12] a) T. R. Strick, J. F. Allemand, D. Bensimon, A. Bensimon, V. Croquette, Science 1996, 271, 1835; b) T. R. Strick, J. F. Allemand, D. Bensimon, V. Croquette, Biophys. J. 1998, 74, 2016.
[13] S. Kim, P. C. Blainey, C. M. Schroeder, X. S. J. N. m. Xie, 2007, 4, 397.
[14] S. Le, R. Liu, C. T. Lim, J. Yan, Methods 2016, 94, 13.
[15] C. Bustamante, J. Marko, E. Siggia, S. Smith, Proc. Nati. Acad. Sci USA 1991, 88, 10009.
[16] M. Mandelkern, J. G. Elias, D. Eden, D. M. Crothers, J. Mol. Biol. 1981, 152, 153.
[17] a) T. Strunz, K. Oroszlan, R. Schäfer, H.-J. J. P. o. t. N. A. o. S. Güntherodt, 1999, 96, 11277; b) K. Hatch, C. Danilowicz, V. Coljee, M. J. P. R. E. Prentiss, 2008, 78, 011920.
[18] G. Neuert, C. Albrecht, E. Pamir, H. E. Gaub, FEBS Lett. 2006, 580, 505.
[19] E. Evans, K. J. B. j. Ritchie, 1997, 72, 1541.
[20] R. Merkel, P. Nassoy, A. Leung, K. Ritchie, E. Evans, Nature 1999, 397, 50.
[21] X. Wang, T. J. S. Ha, 2013, 340, 991.
[22] E. J. A. r. o. b. Evans, b. structure, 2001, 30, 105.
[23] A. B. Patel, S. Allen, M. C. Davies, C. J. Roberts, S. J. Tendler, P. M. Williams, J. Am. Chem. Soc. 2004, 126, 1318.
[24] C. Yuan, A. Chen, P. Kolb, V. T. Moy, Biochemistry 2000, 39, 10219.
[25] V. P. Y. Ma, K. J. S. Salaita, 2019, 1900961.
[26] X. Wang, Z. Rahil, I. T. Li, F. Chowdhury, D. E. Leckband, Y. R. Chemla, T. Ha, Sci. Rep. 2016, 6, 21584.
[27] a) M. T. Woodside, W. M. Behnke-Parks, K. Larizadeh, K. Travers, D. Herschlag, S. M. J. P. o. t. N. A. o. S. Block, 2006, 103, 6190; b) Y. Zhang, C. Ge, C. Zhu, K. J. N. c. Salaita, 2014, 5, 5167.
[28] E. Evans, K. Ritchie, Biophys. J. 1997, 72, 1541.
[29] Mosayebi, A. A. Louis, J. P. Doye, T. E. Ouldridge, ACS Nano 2015, 9, 11993.
[30] P. K. Dutta, Y. Zhang, A. T. Blanchard, C. Ge, M. Rushdi, K. Weiss, C. Zhu, Y. Ke, K. Salaita, Nano Lett. 2018, 18, 4803.
[31] R. Owczarzy, B. G. Moreira, Y. You, M. A. Behlke, J. A. Walder, Biochemistry 2008, 47, 5336.
[32] P. Y. Chiou, A. T. Ohta, M. C. Wu, Nature 2005, 436, 370.
[33] M. Otten, W. Ott, M. A. Jobst, L. F. Milles, T. Verdorfer, D. A. Pippig, M. A. Nash, H. E. Gaub, Nat. Methods 2014, 11, 1127.
[34] S. P. Smith, S. R. Bhalotra, A. L. Brody, B. L. Brown, E. K. Boyda, M. Prentiss, American Journal of Physics 1999, 67, 26.
[35] R. N. Maamari, J. D. Keenan, D. A. Fletcher, T. P. Margolis, Br. J. Ophthalmol. 2014, 98, 438.
[36] a) E. K. Sackmann, A. L. Fulton, D. J. J. N. Beebe, 2014, 507, 181; b) E. Akbari, G. B. Spychalski, J. W. Song, Microcirculation 2017, 24; c) N. Lion, T. C. Rohner, L. Dayon, I. L. Arnaud, E. Damoc, N. Youhnovski, Z. Y. Wu, C. Roussel, J. Josserand, H. Jensen, J. S. Rossier, M. Przybylski, H. H. Girault, Electrophoresis 2003, 24, 3533; d) J. Charmet, P. Arosio, T. P. J. Knowles, J. Mol. Biol. 2018, 430, 565.

### Reference List #3

[1] Polacheck, William J., and Christopher S. Chen, Nat. Methods 2016, 13, 415.
[2] Wang, Xuefeng, and T. Ha, Science 2013, 340, 991.
[3] Zhang, Yun, et al., Nature communications 2014, 5, 1.
[4] P. K. Dutta, Y. Zhang, A. T. Blanchard, C. Ge, M. Rushdi, K. Weiss, C. Zhu, Y. Ke, K. Salaita, Nano Lett. 2018, 18, 4803.
[5] Rothemund, Paul WK, Nature 2006, 440, 297.
[6] Carlos Castro, et al., Nat. Methods 2011, 8, 221.
[7] Wang, Pengfei, et al., Chem 2017, 2, 359.
[8] Akbari, Ehsan, et al. Advanced Materials 2017, 29, 1703632.

1. A microfluidic device for measuring hydrodynamic force between two components, wherein the device comprises:
a. a rigid material, wherein said rigid material comprises at least two channels of varying width that are serially connected; and
b. a first component, wherein said first component is functionally attached to a second component, and further wherein the second component is anchored to the rigid material.

2. (canceled)

3. The device of any one of claim 1, wherein the channels are connected via at least one channel connector.

4. (canceled)

5. The device of claim 1, wherein the device comprises an inlet port and an outlet port, wherein the inlet port is connected to a proximal end of a channel, and the outlet port is connected to a distal end of another channel.

6. (canceled)

7. (canceled)

8. The device of claim 1, wherein the rigid material is a coverslip functionalized with polyethylene glycol, biotin-polyethylene glycol, or a combination thereof.

9. (canceled)

10. The device of claim 1, wherein the channels are made of polydimethylsiloxane.

11. The device of claim 1, wherein the first component is a microbead or a first protein.

12. The device of claim 1, wherein the second component is a first nucleic acid or a microbead.

13. The device of claim 12, wherein the first nucleic acid is single stranded, and is affixed to the coverslip at a proximal end.

14. The device of claim 13, wherein the first nucleic acid can hybridize with a second nucleic acid at a distal end, thereby forming a double stranded nucleic acid molecule.

15. The device of claim 14, wherein the second nucleic acid is tethered to the microbead on its distal end.

16. The device of claim 13, wherein the first nucleic acid is affixed to the coverslip via biotin/streptavidin.

17. The device of claim 14, wherein the second nucleic acid is affixed to the microbead via biotin/streptavidin or via an antibody/antigen interaction.

18. (canceled)

19. (canceled)

20. (canceled)

21. The device of claim 11, wherein the first protein is affixed to the coverslip via biotin/streptavidin.

22. (canceled)

23. (canceled)

24. The device of claim 12, wherein a second protein is affixed to the microbead via biotin/streptavidin.

25. The device of claim 1, wherein the rigid material comprises at least 3 channels.

26. (canceled)

27. (canceled)

28. (canceled)

29. The device of claim 1, wherein at least one channel has a width from 20 to 2500 microns.

30. (canceled)

31. (canceled)

32. (canceled)

33. (canceled)

34. The device of claim 1, wherein the channels have a height from 2 to 150 microns.

35-38. (canceled)

39. A system for measuring molecular interactions between molecules, the system comprising a microfluidic device comprising a rigid material, wherein said rigid material comprises at least two channels of varying width that are serially connected; and a first component, wherein said first component is functionally attached to a second component, and further wherein the second component is anchored to the rigid material, wherein fluid can be forced through the channels, and further wherein hydrodynamic force between the first component and the second component can be measured.

40-44. (canceled)

45. A method of determining strength of molecular interactions between a first molecule and a second molecule comprising:
a. Providing a device, wherein the device comprises
i. a rigid material, wherein said rigid material comprises at least two channels of varying width that are serially connected;
ii. a first component with the first molecule associated therewith; and
iii. a second component, with a second molecule associated therewith; wherein the first component and the second component can associate with each other through the first and second molecules;
b. executing at least one force spectroscopy test;
c. measuring dissociation force between the first and the second molecule, thereby determining the strength of the molecular interaction between the first and the second molecule.

46-54. (canceled)

55. A method of manufacturing a device, the method comprising designing a network of at least two channels; producing transparencies; using the transparencies as masks in photolithography to spin-coat the network of at least two channels onto a wafer to create a microfluidic platform with at least two channels; placing at least one post on the design of at least two channels to form at least one reservoir; casting a prepolymer onto the microfluidic platform; curing the prepolymer; removing the prepolymer from the microfluidic platform; oxidizing the prepolymer in a plasma discharge; and attaching the prepolymer to a rigid material.

56-67. (canceled)
